(19) United States
(12) Patent Application Publication
(10) Pub. No.: US 2026/0265363 A1
(43) Pub. Date: Sep. 10, 2026

(54) TREATMENT OF ASTHMA WITH AN ANTI-INTERLEUKIN-33 ANTIBODY

(71) Applicant: MEDIMMUNE LIMITED, Cambridge (GB)

(72) Inventors: Muhammad Waqas SADIQ, Södertälje (SE); Eulalia Jimenez LOZANO, Madrid (ES)

(21) Appl. No.: 19/106,746

(22) PCT Filed: Aug. 25, 2023
(86) PCT No.: PCT/EP2023/073355

§ 371 Date: Feb. 26, 2025

(30) Foreign Application Priority Data
Aug. 26, 2022 (EP) 22192306.3

(51) Int. Cl. C07K 16/24; A61K 39/00; A61P 11/06

(52) U.S. Cl. CPC C07K 16/244; A61P 11/06; A61K 2039/505; A61K 2039/54; A61K 2039/545; C07K 2317/21; C07K 2317/565; C07K 2317/76

(57) ABSTRACT

The present disclosure relates to methods of treating asthma, particularly by administering an anti-IL-33 antibody or antibody variant thereof in a specified dosing regimen.

### REFERENCE TO SEQUENCE LISTING SUBMITTED ELECTRONICALLY

This application incorporates by reference a Sequence Listing submitted with this application in computer readable form (CRF) as a text file entitled “IL33-400-WO-PCT Sequence Listing” created on Aug. 17, 2023 and having a size of 13,496 bytes.

### CROSS-REFERENCE TO RELATED APPLICATIONS

This application claims benefit of European Patent Application No. 22192306.3 filed Aug. 26, 2022, which is incorporated by reference herein in its entirety for all purposes.

### TECHNICAL FIELD

The present disclosure relates to methods of treating asthma, particularly by administering an anti-IL-33 antibody or antibody variant thereof.

### BACKGROUND

Asthma is a chronic inflammatory disease of the airways characterised by bronchial hyperreactivity and reversible airflow limitation. International treatment guidelines for asthma recommend ICS as first line therapy (GINA 2020). For individuals who are symptomatic on medium dose ICS monotherapy, step-up therapy with LABA is the recommended next treatment option followed by other controller therapies including leukotriene receptor antagonists, theophylline, and oral corticosteroids. ‘Biologic agents’ (eg, omalizumab and benralizumab) that inhibit specific molecular targets such as IgE or Th2 cytokines and their respective receptors are reserved for those with severe uncontrolled asthma. Furthermore, all currently approved asthma controller therapies have little or no impact on the natural history of the disease (ie, most people with moderate-severe asthma need life-long therapy). There is a clear unmet need for asthma therapies that not only better control symptoms, but lead to disease modification.

Numerous studies have shown a key role for IL-33 in asthma. The genes encoding IL-33 and ST2/ILIRL1 have been identified as major susceptibility loci for human asthma in several genome wide association studies (Bonnelykke et al 2014; Gudbjartsson et al 2009; Hirota et al 2011; Moffat et al 2010; Shrine et al 2019; Torgerson et al 2011; Wan et al 2012) and were among the few genes reproducibly found to be associated with asthma across diverse ethnic groups. In addition, a rare IL-33 loss-of-function mutation has been recently described that reduces blood eosinophil counts and protects from asthma (Smith et al 2017). The protective effect remained, although weaker, after correcting for eosinophil counts, suggesting that IL-33 may influence asthma risk in part by controlling blood eosinophil count, and also through additional biological pathways (Mousas et al 2017). Two Phase II studies support a role for anti-IL-33 therapy in asthma treatment. In a proof-of-concept study involving adult patients with moderate-to-severe asthma, REGN3500 monotherapy (ICS and LABA maintenance therapy were withdrawn during the trial) met the primary endpoint of improvement in loss of asthma control and significantly improved lung function compared to placebo, meeting a key secondary endpoint (NCT03387852). In a separate Phase II clinical study, a single IV dose of etokimab was administered to patients with uncontrolled moderate-to-severe asthma despite being on treatment with high dose combination ICS plus LABA therapy. Compared with a placebo group, asthmatics treated with etokimab had a significant improvement in lung function (FEV1) and a reduction in blood eosinophils (NCT03469934).

Tozorakimab is a human IgG1 mAb that binds to human IL-33. Tozorakimab binds full length and mature forms of human IL-33 with exceptionally high affinity and prevents IL-33 binding to soluble (sST2) and membrane-bound forms of ST2 (also known as IL-IRL1) receptor. Several clinical and non-clinical studies point to the IL-33/ST2 signaling axis playing a key role in the pathogenesis of asthma.

The present disclosure describes a randomized, double-blind, placebo-controlled study to assess the efficacy and safety of tozorakimab in adult participants with uncontrolled moderate-to-severe asthma.

### SUMMARY OF THE DISCLOSURE

The present disclosure provides methods for treating asthma. The methods disclosed herein comprise administration of anti-IL-33 antibodies or antibody variants thereof.

The examples show that the anti-IL33 antibody tozorakimab (also known as MEDI3506 and 33-670087_7B) may be effective in the treatment of chronic obstructive pulmonary disorder (COPD), validating the pharmacological activity of tozorakimab with respect to inhibiting IL-33 activity. As outlined in the background, at least two clinical studies have validated that IL-33 inhibition can be an effective treatment for asthma. Thus, it is credible that the tozorakimab dosing regimens disclosed herein that lead to effective target inhibition in the lung would work well for both the treatment of COPD and asthma.

In one aspect, the disclosure provides a method of treating asthma in a subject comprising administering a therapeutically effective amount of an anti-IL-33 antibody or antibody variant thereof in a dose of from about 300 to about 600 mg at an interval of every 4 weeks (Q4W) or 8 weeks (Q8W), wherein the anti-IL-33 antibody comprises: a heavy chain variable region comprising a VHCDR1 having the sequence as set forth in SEQ ID NO: 1, a VHCDR2 having the sequence of SEQ ID NO: 2, a VHCDR3 having the sequence of SEQ ID NO: 3; and a light chain variable region comprising a VLCDR1 having the sequence of SEQ ID NO: 5, a VLCDR2 having the sequence of SEQ ID NO: 6, and a VLCDR3 having the sequence of SEQ ID NO: 7.

In another aspect, the disclosure provides a method of treating asthma in a subject comprising administering a therapeutically effective amount of an anti-IL-33 antibody or antibody variant thereof in a dose of about 150 mg at an interval of every 4 weeks (Q4W), wherein the anti-IL-33 antibody comprises: a heavy chain variable region comprising a VHCDR1 having the sequence as set forth in SEQ ID NO: 1, a VHCDR2 having the sequence of SEQ ID NO: 2, a VHCDR3 having the sequence of SEQ ID NO: 3; and a light chain variable region comprising a VLCDR1 having the sequence of SEQ ID NO: 5, a VLCDR2 having the sequence of SEQ ID NO: 6, and a VLCDR3 having the sequence of SEQ ID NO: 7.

In another aspect, the disclosure provides a method of treating asthma in a subject comprising administering a therapeutically effective amount of an anti-IL-33 antibody or antibody variant thereof in a dose effective to achieve at least 80% inhibition of IL-33 in the lung or epithelial lining fluid (ELF), wherein the anti-IL-33 antibody comprises: a heavy chain variable region comprising a VHCDR1 having the sequence as set forth in SEQ ID NO:1, a VHCDR2 having the sequence of SEQ ID NO: 2, a VHCDR3 having the sequence of SEQ ID NO: 3; and a light chain variable region comprising a VLCDR1 having the sequence of SEQ ID NO: 5, a VLCDR2 having the sequence of SEQ ID NO: 6, and a VLCDR3 having the sequence of SEQ ID NO: 7.

In some instances, the dose is effective to achieve at least about 90%, optionally at least 95%, inhibition of IL-33 in the lung.

In some instances, the dose is about 300 to about 600 mg at an interval of every 2 weeks (Q2W), every 4 weeks (Q4W) or 8 weeks (Q8W). In some instances, the dose is about 300 mg Q8W. In some instances, the dose is about 300 mg Q4W. In some instances, the dose is about 150 mg Q4W. In some instances, the dose is about 300 mg Q2W.

In some instances, the anti-IL-33 antibody or antibody variant thereof is selected from: human antibody, a humanized antibody, a chimeric antibody, a monoclonal antibody, a recombinant antibody, an antigen-binding antibody fragment, a single chain antibody, a monomeric antibody, a diabody, a triabody, tetrabody, a Fab fragment, an IgG1 antibody, an IgG2 antibody, an IgG3 antibody, and an IgG4 antibody.

In some instances, the anti-IL-33 antibody or antibody variant thereof is an IgG1.

In some instances, the anti-IL-33 antibody or antibody variant thereof is a human antibody.

In some instances, the anti-IL-33 antibody or antibody variant thereof comprises a VH domain at least 95%, 90% or 85% identical to the sequence set forth in SEQ ID NO: 4 and a VL domain at least 95%, 90% or 85% identical to the sequence set forth in SEQ ID NO: 8.

In some instances, the anti-IL-33 antibody comprises a VH domain sequence as set forth in SEQ ID NO: 4 and a VL domain sequence as set forth in SEQ ID NO:8.

In some instances, the anti-IL-33 antibody comprises a light chain sequence as set forth in SEQ ID NO: 9 and a heavy chain sequence as set forth in SEQ ID NO: 10.

In some instances, the anti-IL-33 antibody variant has the same pharmacokinetic (pK) characteristics as tozorakimab in humans.

In some instances, the anti-IL-33 antibody is tozorakimab.

In some instances, the administration is subcutaneous.

In some instances, for any of the preceding aspects, the anti-IL-33 antibody or antibody variant thereof is administer for a period of at least 12 weeks. In some instances, for any of the preceding aspects, the anti-IL-33 antibody or antibody variant thereof is administer for a period of at least 24 weeks. In some instances, for any of the preceding aspects, the anti-IL-33 antibody or antibody variant thereof is administer for a period of at least 52 weeks.

### BRIEF DESCRIPTION OF DRAWINGS

FIG. 1A shows IL-33/tozorakimab complex amount measured in serum of healthy participants from Part I of NCT03096795

FIG. 1B shows IL-33/sST2 complex amount measured in serum of healthy participants from Part I of NCT03096795

FIG. 1C shows IL-33/tozorakimab complex amount measured in serum of participants with COPD from Part II of NCT03096795

FIG. 1D shows IL-33/sST2 complex amount measured in serum of participants with COPD from Part II of NCT03096795

FIG. 2A shows nasal mucosal lining fluid levels of free IL-33^{red} plus IL-33red/tozorakimab measured at day 29 in the 300 mg MAD cohort

FIG. 2B shows nasal mucosal lining fluid levels of free IL-33^{red} measured at day 29 in the 300 mg MAD cohort

FIG. 2C shows nasal mucosal lining fluid levels of free IL-33^{ox} measured at day 29 in the 300 mg MAD cohort

FIG. 3 shows tozorakimab inhibits ex vivo IL-33 challenge in whole blood from healthy participants

FIG. 4A shows serum levels of IL-5 at days 1, 14 and 28 in participants in the 300 mg MAD cohort (Placebo, n=6; tozorakimab (n=6). The plot shows mean±SEM. A mixed-effect longitudinal model was used to generate p values comparing the trajectories of the biomarkers between tozorakimab and placebo. (p=0.0037)

FIG. 4B shows serum levels of IL-13 at days 1, 14 and 28 in participants in the 300 mg MAD cohort (Placebo, n=6; tozorakimab (n=6). The plot shows mean±SEM. A mixed-effect longitudinal model was used to generate p values comparing the trajectories of the biomarkers between tozorakimab and placebo. (p=0.034)

FIG. 4C shows serum levels of eosinophils at days 1, 14 and 28 in participants in the 300 mg MAD cohort (Placebo, n=6; tozorakimab (n=6). The plot shows mean±SEM. A mixed-effect longitudinal model was used to generate p values comparing the trajectories of the biomarkers between tozorakimab and placebo. (p=0.0023)

FIG. 5 shows that Alternaria alternata induces rapid IL-33 release in the bronchoalveolar lavage fluid (BALF) in humanised IL-33 mice

FIG. 6 shows tozorakimab inhibits ALT-induced BALF IL-5 in humanized IL-33 mice. Test substances were dosed intranasally at −24 hours prior to challenge with ALT. BALF was harvested at 24 hours post ALT challenge and analysed for presence of IL-5. Significant effect of test substances was determined using one-way ANOVA with Bonferroni's multiple comparisons test. *** p<0.001, ** p<0.01 (n=4)

FIG. 7A shows scratch wound repair in normal human bronchial epithelial cells upon treatment with wildtype IL-33 (IL-33), oxIL-33, and oxIL-33+anti-ST2 antibody

FIG. 7B shows quantification of the % wound closure from scratch wound assays described in FIG. 3A

FIG. 8 shows scratch wound impairment was also seen in bronchial epithelial cells obtained from COPD subjects

FIG. 9 shows % scratch closure in A549 cells with increasing concentrations of tozorakimab and anti-TSLP antibody

FIG. 10 is the PK/PD target engagement model description

FIG. 11 shows the prediction of tozorakimab systemic concentrations vs the observed tozorakimab systemic concentrations from Ph1 dose cohorts

FIG. 12 shows the prediction of tozorakimab: IL-33 complex formation vs the observed tozorakimab: IL-33 complex formation from Ph1 dose cohorts

FIG. 13 shows the prediction of IL-33: sST2 complex reduction vs the observed IL-33: sST2 complex reduction from Ph1 dose cohorts

FIG. 14 shows the IL-33 inhibition predicted from dose response for IL33/sST2 complex inhibition in blood (Q2W-top line; Q4W-middle line; Q6W-bottom line)

FIG. 15 shows the predicted suppression of IL-33 in the lung at trough with tozorakimab (Q4W-top line; Q8W-bottom line)

FIG. 16 shows the predicted tozorakimab serum concentration following 300 mg Q4W (higher line) and 300 mg Q8W (lower line). The serum concentrations required for 60%, 80% and 90% identified by Alternaria mouse model are indicated by dashed lines

FIG. 17 shows the predicted tozorakimab serum concentration following 300 mg Q4W (higher line) and 150 mg Q4W (lower line). The serum concentration threshold for response in the scratch wound closure assay in shown as a dashed line

FIG. 18 also shows the prediction of tozorakimab systemic concentrations vs the observed tozorakimab systemic concentrations from Ph1 dose cohorts

FIG. 19 also shows the prediction of IL-33: sST2 complex reduction vs the observed IL-33: sST2 complex reduction from Ph1 dose cohorts

### DETAILED DESCRIPTION

The term “about” or “approximately” means an acceptable error for a particular value as determined by one of ordinary skill in the art, which depends in part on how thevalue is measured or determined. In certain embodiments, the term “about” or “approximately” means within 1, 2, 3, or 4 standard deviations. In certain embodiments, the term “about” or “approximately” means within 30%, 25%, 20%, 15%, 1 0%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

### Asthma

Asthma is a chronic inflammatory disease of the airways affecting 1-18% of the population in different countries and is characterized by bronchial hyperreactivity and reversible airflow limitation. It is defined by the history of respiratory symptoms such as wheeze, shortness of breath, chest tightness and cough. The etiology of asthma is thought to be multi-factorial and there are recognizable clusters of demographic, clinical and/or pathophysiological phenotypes. In patients with more severe phenotypes, some phenotype-guided treatment are available. However, no strong relationship between pathological symptoms and clinical presentations and response to therapies have been established.

Different asthma subtypes have been identified, including allergic asthma, non-allergic asthma, late-onset asthma (which typically tends to be non-allergic), asthma with persistent airflow limitation (which is linked to airway wall remodeling, leading to a long-standing, persistent, irreversible airflow limitation), and asthma with obesity (which is typically linked to a non/low eosinophilic mechanism of action).

There are different levels of asthma severity, which are currently assessed retrospectively from the level of treatment required to control symptoms and exacerbations. The severity index comprises three main groups: mild asthma, moderate asthma, and severe asthma. Severity of asthma is defined on the GINA scale by the level of treatment required to gain adequate control of symptoms. The GINA scale is defined in the “Pocket Guide for Asthma Management and Prevention,” Global Initiative for Asthma; 2019.

In certain instances, the subjects suitable for treatment by the methods disclosed herein are suffering from moderate asthma, severe asthma, or moderate-to-severe asthma. In some instances, the subjects suitable for treatment by the methods disclosed herein are not well-controlled on controller or reliever standard of care therapies defined in steps 1 and 2 of the GINA scale.

In certain instances, the subjects suitable for treatment by the methods disclosed herein are suffering from moderate asthma uncontrolled on standard of care therapy, severe asthma uncontrolled on standard of care therapy, and moderate-to-severe asthma uncontrolled on standard of care therapy. Standard of care (SOC) therapy is as defined in the GINA scale.

Asthma may be diagnosed or assessed by a number of different measures, including:

Airway inflammation evaluated using a standardized single-breath Fraction of Exhaled Nitric Oxide (FeNO) (ATS, Am J Repir Crit Care Med. 171 (8): 912-30, 2005). FeNO has not been established for confirming asthma diagnosis but elevated FeNO has been associated with asthma characterized by a Type 2 airway inflammation.

Determining the atopic status. This can be identified by a skin prick test with common environmental alleges or by measuring the level of specific IgE in serum. As with FeNO, allergy tests do not rule in or rule out a diagnosis of asthma but the presence of atopy increases the probability that a patient with respiratory symptoms has allergic asthma.

Bronchial provocation testing. These tests monitor variable airflow limitation to assess airway hyperresponsiveness (AHR). Subjects can be challenged with chemical agents such as methacholine. Such tests are moderately sensitive to the diagnosis of asthma.

The Asthma Control Questionnaire (ACQ) 6 is a patient-reported questionnaire assessing asthma symptoms (i.e., night-time waking, symptoms on waking, activity limitation, shortness of breath, wheezing) and daily rescue bronchodilator use and FEV1 (Juniper et al, October 1999). The ACQ-6 is a shortened version of the ACQ that omits the FEV1 measurement from the original ACQ score. Questions are weighted equally and scored from 0 (totally controlled) to 6 (severely uncontrolled). The mean ACQ score is the mean of the responses. Mean scores of 0.75 indicate well-controlled asthma, scores between 0.75 and 1.5 indicate partly-controlled asthma, and a score>1.5 indicates uncontrolled asthma (Juniper et al, Respir Med. 1 00 (4): 616-21, 2006). Individual changes of at least 0.5 are considered to be clinically meaningful (Juniper et al, Respir Med. 99 (5): 553-8, 2005). In certain instances, the patients suitable for treatment with the methods contemplated herein have an ACQ-6 score of ≥1.5, before first dose with the therapy contemplated herein.

Spirometry is performed according to ATS/European Respiratory Society (ERS) guidelines (Miller et al, Eur Respir J. 26(1): 153-61, 2005). For example, multiple forced expiratory efforts (at least 3 but no more than 8) is performed at each spirometry session and the 2 best efforts that meet ATS/ERS acceptability and reproducibility criteria are recorded. The best efforts will be based on the highest FEV1. The maximum fluvial exhalation volume (FEV1) of the 2 best efforts will be used for the analysis. Both the absolute measurement (for FEV1 and forced vital capacity (FVC)) and the percentage of predicted normal value will be recorded using appropriate reference values. The highest FVC will be reported regardless of the effort in which it occurred (even if the effort did not result in the highest FEV₁). In certain instances, the patients suitable for treatment with the methods contemplated herein have a morning pre-BD FEV₁ of greater than or equal to 40% predicted normal and greater than 1 L, but a pre-BD FEV₁ of less than 85% predicted normal.

Post-bronchodilator (Post-BD) spirometry testing is assessed after the subject has performed pre-BD spirometry. Maximal bronchodilation is induced using a short-acting beta agonist (SABA) such as albuterol (90 1-1g metered dose) or salbutamol (1 00 1-1g metered dose) or equivalent with a spacer device for a maximum of 8 total puffs (Sorkness et al, J Appl Physiol. 1 04 (2): 394-403, 2008). The highest pre- and post-BD FEV1 obtained after 4, 6, or 8 puffs is used to determine reversibility and for analysis. Reversibility algorithm is as follows:

$$\% Reversibility = ( post - BD FEV 1 - pre - BD FEV 1 ) \times 1 \; 00 / pre - BD FEV 1$$

In certain instances, the patients suitable for treatment with the methods contemplated herein have a % Reversibility of greater than or equal to 12% and greater than or equal to 200 ml (for example, 15 to 60 min after administration of 4 puffs of albuterol/salbutamol).

In some instances, the subjects suitable for treatment by the methods disclosed herein are diagnosed with early-onset asthma. As defined herein “early-onset” asthma refers to a subject diagnosed with asthma before the age of 25, preferably before the age of 18. Diagnosis may be made by a clinician, for example, using any one of a number of well-known methods for diagnosing asthma. It is to be understood that methods disclosed herein for use in early-onset asthma sufferers are not confined to subjects below the age of 25 (or 18, even). For example, the methods may be used in an adult (defined herein as someone above the age of 18) but who has been suffering from asthma since before the age of 25.

### Administration Regimens

The present disclosure relates to dosage regimens of an anti-IL-33 antibody or antibody variant, which finds particular effectiveness in the treatment of asthma. A dosage regimen is made up of one or more doses of a controlled size, administered throughout a treatment window. Where there are more than one dose, the doses are separated by a dosing interval. The anti-IL-33 antibody or antibody variant is administered in a therapeutically effective amount. As used herein, an “effective amount” or “therapeutically effective amount” of an agent, e.g., a pharmaceutical formulation comprising an IL-33 antibody, refers to an amount effective, at dosages and for periods of time necessary, to achieve the desired therapeutic or prophylactic result.

The size of the dose of anti-IL-33 antibody or antibody variant thereof may be expressed in terms of weight of the anti-IL-33 antibody or antibody variant. In certain instances, the anti-IL-33 antibody or antibody variant thereof is administered in a dose of about 400 to about 800 mg, about 450 to about 750 mg, about 500 to about 700 mg, about 510 to about 690 mg, about 520 to about 680 mg, about 530 to about 670 mg, about 540 to about 660 mg, about 550 to about 650 mg, about 560 to about 640 mg, about 570 to about 630 mg, about 580 to about 620 mg, about 590 to about 630 mg, or about 600 mg.

In some instances, the dose is 600 mg. In some instances, the anti-IL-33 antibody or antibody variant thereof is formulated for subcutaneous injection at 150 mg/mL, such that a 600 mg dose is administered as a 4 mL treatment. A 600 mg dose of the anti-IL-33 antibody or antibody variant thereof may be administered as two concurrent 300 mg dosages. As used herein, “concurrent” doses refer to doses which are administered simultaneously, or sequentially with no or only a minimal time period (e.g. less than 1 hour, less than 30 minutes, less than 15 minutes, less than 5 minutes) separating them.

In some instances, the anti-IL-33 antibody or antibody variant thereof is administered in a dose of about 200 to about 400 mg, about 250 to about 350 mg, about 260 to about 340 mg, about 270 to about 330 mg, about 280 to about 320 mg, about 290 to about 310 mg, about 295 to about 305 mg or about 300 mg.

In some instances, the dose is 300 mg. In some instances, the anti-IL-33 antibody or antibody variant thereof is formulated for subcutaneous injection at 150 mg/mL, such that a 300 mg dose is administered as a 2 mL treatment. In some instances, a 300 mg dose of the anti-IL-33 antibody or antibody variant thereof may be administered as two concurrent 150 mg dosages. As used herein, “concurrent” doses refer to doses which are administered simultaneously, or sequentially with no or only a minimal time period (e.g. less than 1 hour, less than 30 minutes, less than 15 minutes, less than 5 minutes) separating them.

In some instances, the dose is 150 mg. In some instances, the anti-IL-33 antibody or antibody variant thereof is formulated for subcutaneous injection at 150 mg/mL, such that a 150 mg dose is administered as a 1 mL treatment.

The size of the dose of an anti-IL-33 antibody or antibody variant thereof may be expressed in terms of the plasma drug concentration provided by the dose, as the amount of active compound manipulated so as to provide a plasma drug concentration of a certain level. By varying the amount, bioavailability, or timing/frequency of the antibody or variant administered, the skilled person can control the plasma concentration in the subject. As plasma concentrations vary across time with drug uptake and clearance, they may be expressed in various standardised ways—for example as a maximum, minimum (trough) or across time.

In some instances, the dose is selected so as to provide a Cmax,ss (the observed maximum concentration at steady state) of between about 20 and about 50 μg/mL, between about 25 and about 45 μg/mL, between about 30 and about 40 g/mL, between about 35 and about 40 μg/mL, or about 37 μg/mL In some instances, the Cmaz.ss is that observed during the dosing period. In this context, the “dosing period” refers to the time between two consecutive doses.

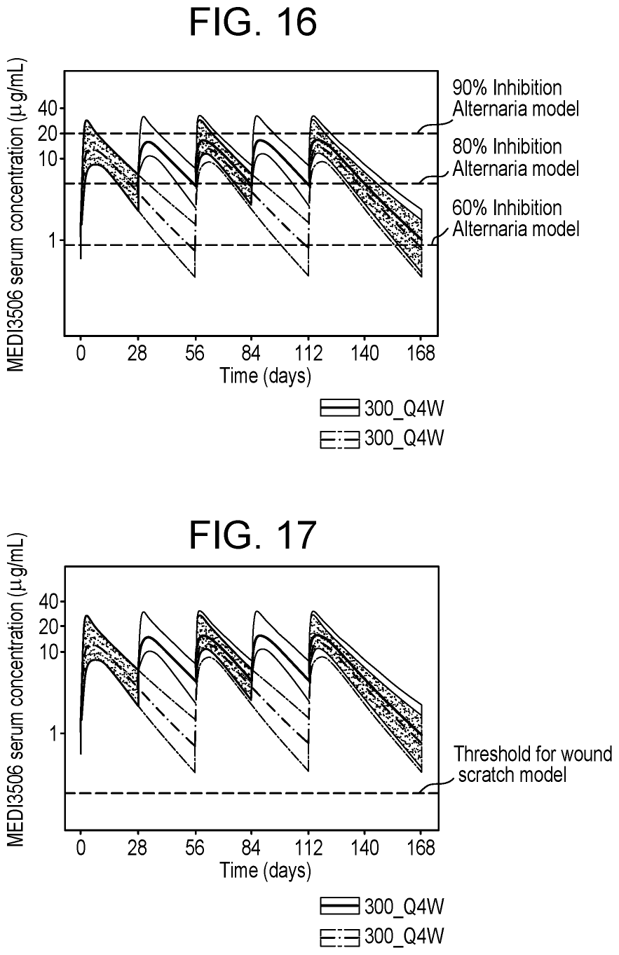

The examples show that a 300 mg Q4W or Q8W dosing regimen of MEDI3506 are predicted to achieve serum concentrations necessary for inhibition of both the redIL-33: ST2 signalling axis and the oxIL-33: RAGE/EGFR signalling axis, achieving sustained, dual pathway inhibition (FIGS. 16 and 17). The MEDI3506 serum concentration can be measured (and therefore used to determine C_{max.ss}) anti-drug antibody reagents in a suitable assay format to capture and detect MEDI3506 from a biological sample (e.g. blood). In some instances, the assay may use an anti-IgG1 capture mAb and a stabilised MEDI3506 antigen labelled with a detectable marker. The detectable marker can be quantified in order to determine the concentration of MEDI3506. In some instances, the MEDI3506 antigen (IL-33) may stabilised in a reduced form, for example, by mutating one or more cysteine residues to serine to prevent redIL-33 conversion to the oxidised form (oxIL-33) through disulphide bond formation. Assays and platforms suitable for the detection of serum biomarkers are well known to the skilled person.

In some instances, the dose is selected so as to provide a Cmax,ss of between about 10 and about 35 μg/mL, between about 15 and about 30 μg/mL, between about 15 and about 30 μg/mL, between about 15 and about 25 μg/mL, about 15 to about 20 μg/mL, or about 18.6 μg/mL.

In some instances, the anti-IL-33 antibody or antibody variant thereof is administered in a dose selected so as to provide an area under the plasma concentration-time curve throughout a dosing period (AUC).

In some instances, the dose is selected so as to provide an AUC of between about 400 and about 800 μg· day/mL, between about 500 and about 750 μg· day/mL, between about 600 and about 700 μg· day/mL, between about 600 and about 650 μg· day/mL, between about 600 and about 620 μg· day/mL, between about 610 and about 620 μg· day/mL, or about 616 μg· day/mL over the dosing period.

In some instances, the dose is selected so as to provide an AUC of between about 200 and about 515 μg· day/mL, between about 250 and about 500 μg· day/mL, between about 300 and about 450 μg· day/mL, between about 300 and about 350 μg· day/mL, or about 323 μg· day/mL over the dosing period.

In some instances, the dose is selected so as to provide an AUC of between about 100 and about 300 μg· day/mL, between about 100 and about 250 μg· day/mL, between about 100 and about 200 μg· day/mL, between about 150 and about 200 μg· day/mL, or about 161.5 μg· day/mL over the dosing period.

Administration of the anti-IL-33 antibody or antibody variant thereof is performed as multiple doses separated by a dosing interval. In some instances, the dosing interval is 2 weeks (14 days), 3 weeks (21 days), 4 weeks (28 days) or 5 weeks (35 days). In some embodiments, the dosing interval is 4 weeks (28 days). In some instances, the dosing interval is about 4 weeks (i.e. 28±4 days). In some instances, the dosing interval is about 8 weeks (i.e. 56±4 days).

In some instances, a dose may be administered across multiple days, for example, as two or more sub-doses. As used herein, a “sub-dose” is a fractional quantity of a dose of therapeutic, such that the total quantity of therapeutic administered in sub-doses is equal to that in the dose. Any fractional quantity may be used, for example such that two, three, four, five or more sub-doses comprise a single dose. In some instances, a dose may be administered as two or more sub-doses separated by a period of 1, 2, 3, 4, 5, or 6 days. In some instances, a dose may be administered as two or more sub-doses separated by a period of 1, 2, or 3 weeks. Sub-doses may be administered on two, three, four or more consecutive days. Sub-doses which make up a dose may be of equal size, or may differ in size, so long as their total is equal to the dose.

Therefore, as used herein, a dose of 600 mg with a 4 week dosing window (Q4W) may be substituted for 150 mg administered weekly (Q1W), 300 mg administered every 2 weeks (Q2W), or 450 mg administered every 3 weeks (Q3W), all of which provide a dosing regimen equivalent to 600 mg every 4 weeks. Thus, in some instances, the dose is about 300 mg Q2W. A dose of 300 mg with a 4 week dosing window (Q4W) may be substituted for 150 mg administered every two weeks (Q2W) or 75 mg administered weekly (Q1W). A dose of 300 mg with an 8 week dosing window (Q8W) may be substituted for 150 mg administered every four weeks (Q4W) or 75 mg administered every two weeks (Q2W), or 37.5 mg administered every week (Q1W).

When the dosing interval is expressed as a number of weeks, a margin of error is permissible such that a week may be expressed as 7 days #1 day. In some embodiments, a week may be expressed as 7 days±0.5 days, 7 days±0.25 days, or exactly 7 days. Where the dosing interval is multiple weeks, the margins of error in each week may be combined. For example, in some instances, the dosing interval is 4 weeks±4 days. In some instances, the dosing interval is 4 weeks±3 days. In some embodiments, the dosing interval is 4 weeks±2 days. In some embodiments, the dosing interval is 4 weeks±1 day. In some instances, the dosing interval is exactly 4 weeks. In some instances, the dosing interval is 8 weeks±4 days. In some instances, the dosing interval is 8 weeks±3 days. In some instances, the dosing interval is 8 weeks±2 days. In some instances, the dosing interval is 8 weeks±1 day. In some instances, the dosing interval is exactly 8 weeks.

In some instances, the anti-IL-33 antibody or antibody variant thereof is administered during a “treatment window”, which as used herein refers to a period commencing at the administration of the first dose and running until the final dose of the anti-IL-33 antibody or antibody variant thereof is administered. The date the first dose is administered is referred to as “Day 1” of “Week 0”, with Week 1 commencing 7 days later, Week 2 commencing 7 days after that, and so on. In some embodiments, the treatment window is 12 weeks long (i.e. running from Week 0 to Week 12). In some embodiments, the treatment window is 16 weeks long (i.e. running from Week 0 to Week 15) and the dosing interval is 4 weeks, such that a total of 4 doses are administered (on Week 0, 4, 8 and 12 respectively). In some embodiments, the treatment window is 12 weeks long, and the dosing interval is 4 weeks, such that doses are administered Days 1 (Week 0), 29±4 (Week 4), 57±4 (Week 8), and 85±4 (Week 12).

In some instances, the treatment window is 12 weeks, 14 weeks, 16 weeks, 18 weeks, 20 weeks, 22 weeks, 24 weeks, 26 weeks, 28 weeks, 30 weeks, 32 weeks, 34 weeks, 36 weeks, 38 weeks, 40 weeks, 42 weeks, 44 weeks, 46 weeks, 48 weeks, 50 weeks, 52 weeks or more. In some instances, the treatment window is 52 weeks or more. In some instances, the treatment window is 48 weeks or more.

In some instances, the anti-IL-33 antibody or antibody variant thereof is administered at about 300 mg Q4W. The examples show that administration of MEDI3506 300 mg Q4W is predicted to achieve approximately 94% depletion (target engagement) in the lung. This level of target engagement is potentially greater than that predicted to be achieved for the anti-IL-33 antibody itepekimab, in which a recent Phase II study in COPD resulted in a marked reduction of COPD exacerbations as well as a significant improvement of FEV1 in former smokers (Rabe et al 2021). Itepekimab has a significantly longer reported half-life compared to MEDI3506 (The itepekimab t1/2, which also known as SAR440340 and REGN3500, is reported as 30 days in US2021/0000949-see therein). Such an administration is also suitable for the treatment of asthma, as depletion is desirable for the treatment of both conditions.

In some instances, the anti-IL-33 antibody or antibody variant thereof is administered at about 300 mg Q8W. The examples show that administration of MEDI3506 300 mg Q8W (i.e., with a twice as long dosing window) is predicted to provide approximately 83% inhibition of IL 33 at trough in lung tissue; therefore, it could provide an adequate efficacy in asthma with a more convenient dose frequency for patients compared with Q4W.

In some instances, therefore, the IL-33 antibody or antibody variant thereof is administered at a dose that achieves at least 80%, 85% or 90% target engagement in the lung. In some instances, the dose achieves at least 90% target engagement in the lung. In some instances, the dose achieves at least 91%, 92%, 93% or 94% target engagement in the lung. In some instances the % target engagement is achieved at trough concentration.

### Anti-IL-33 Antibodies

The therapies described herein relate to anti-IL-33 antibodies, and variants and fragments thereof.

Interleukin-33 (IL-33) is a member of the interleukin-1 (IL-1) cytokine family that is encoded by the IL33 gene. IL-33 is constitutively expressed in multiple cell types, including structural cells, such as smooth muscle, epithelial, and endothelial cells. It has been reported that IL-33 expression can also be induced by inflammatory factors in macrophages and dendritic cells. Cellular stress caused by environmental triggers, such as allergens, toxins, and pathogens, and mechanistic insult can lead to IL-33 release. Free IL-33 associates with a heterodimeric IL-33 receptor complex composed of suppression of tumorigenicity 2 (ST2) protein and interleukin-1 receptor accessory protein (IL-1 RAcP) to activate the AP-1 and NF-κB pathways through the adaptor protein myeloid differentiation primary response 88 (MyD88) and possibly MyD88-adapter-like (Mal) protein. IL-33 stimulates numerous cell types, including innate lymphoid type II cells (ILC2), mast cells, basophils, eosinophils, and dendritic cells, to promote an immune response.

The terms “interleukin 1 receptor-like 1 (ILIRL1)” and “ST2,” used interchangeably herein, refer to any native ST2 from any vertebrate source, including mammals such as primates (e.g., humans) and rodents (e.g., mice and rats), unless otherwise indicated. ST2 is also referred to in the art as DER4, T1, and FIT-1. The term encompasses “full-length,” unprocessed ST2, as well as any form of ST2 that results from processing in the cell. At least four isoforms of ST2 are known in the art, including soluble (sST2, also known as IL 1 RL 1-a) and transmembrane (ST2L, also known as IL 1 RL 1-b), which arise from differential mRNA expression from a dual promoter system, and ST2V and ST2LV, which arise from alternative splicing. The domain structure of ST2L includes three extracellular immunoglobulin-like C2 domains, a transmembrane domain, and a cytoplasmic Toll/Interleukin-1 receptor (TIR) domain. sST2 lacks the transmembrane and cytoplasmic domains contained within ST2L and includes a unique 9 amino acid (a.a.)C-terminal sequence (see, e.g., Kakkar et al. Nat. Rev. Drug Disc.40 7:827-840, 2008). sST2 can function as a decoy receptor to inhibit soluble IL-33. The term also encompasses naturally occurring variants of ST2, e.g., splice variants (e.g., ST2V, which lacks the third immunoglobulin motif and has a unique hydrophobic tail, and ST2LV, which lacks the transmembrane domain of ST2L) or allelic variants (e.g., variants that are protective against asthma risk or that confer asthma risk as described herein). The amino acid sequence of an exemplary human

ST2 can be found, for example, under UniProtKB accession number 001638. ST2 is a part of the IL-33 receptor along with the co-receptor protein IL-1 RAcP. Binding of IL-33 to ST2 and the co-receptor interleukin-1 receptor accessory protein (IL-1 RAcP) forms a 1:1:1 ternary signaling complex to promote downstream signal transduction (Lingel et al. Structure 17 (10): 1398-1410,2009, and Liu et al. Proc. Nat. Acad. Sci. 11 0 (37): 14918-14924, 2013).

It is contemplated that antibodies or antibody variants that specifically bind to and inhibit components of the IL-33/ST2 signaling axis may be useful for the treatment of asthma.

“Antibody” is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

It is particularly contemplated that anti-IL33 antibodies or antibody variants, i.e., antibodies that bind specifically to and inhibit/neutralize IL-33, are effective in the treatment of asthma. In some instances, the antibody may be monoclonal (MAbs); recombinant; chimeric; humanized, such as complementarity-determining region (CDR)-grafted; human; antibody variants, including single chain; and/or bispecific; as well as fragments; variants; or derivatives thereof. Antibody fragments include those portions of the antibody that bind to an epitope on the polypeptide of interest. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Monoclonal antibodies may be modified for use as therapeutics or diagnostics. “Monoclonal antibody” or “monoclonal antibody composition” as used herein refers to polypeptides, including antibodies, bispecific antibodies, etc., that have substantially identical amino acid sequence or are derived from the same genetic source. This term also includes preparations of antibody molecules of single molecular composition. A monoclonal antibody composition displays a single binding specificity and affinity for a particular epitope.

One instance is a “chimeric” antibody in which a portion of the heavy (H) and/or light (L) chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity. See U.S. Pat. No. 4,816,567; Morrison et al., 1985, Proc. Natl. Acad. Sci. 81:6851-55.

In another instance, a monoclonal antibody is a “humanized” antibody. Methods for humanizing non-human antibodies are well known in the art. See U.S. Pat. Nos. 5,585,089 and 5,693,762. Generally, a humanized antibody has one or more amino acid residues introduced into it from a source that is non-human. Humanization can be performed, for example, using methods described in the art (Jones et al., 1986, Nature 321:522-25; Riechmann et al., 1998, Nature 332:323-27; Verhoeyen et al., 1988, Science 239:1534-36), by substituting at least a portion of a rodent complementarity-determining region for the corresponding regions of a human antibody.

Also contemplated are human antibodies and antibody variants (including antibody fragments) that bind to IL-33. Using transgenic animals (e.g., mice) that are capable of producing a repertoire of human antibodies in the absence of endogenous immunoglobulin production such antibodies are produced by immunization with a polypeptide antigen (i.e., having at least 6 contiguous amino acids), optionally conjugated to a carrier. See, e.g., Jakobovits et al., 1993, Proc. Natl. Acad. Sci. 90:2551-55; Jakobovits et al., 1993, Nature 362:255-58; Bruggermann et al., 1993, Year in Immuno. 7:33. See also PCT App. Nos. PCT/US96/05928 and PCT/US93/06926. Additional methods are described in U.S. Pat. No. 5,545,807, PCT App. Nos. PCT/US91/245 and PCT/GB89/01207, and in European Patent Nos. 54607381 and 546073A 1. Human antibodies can also be produced by the expression of recombinant DNA in host cells or by expression in hybridoma cells as described herein.

Chimeric, CDR grafted, and humanized antibodies and/or antibody variants are typically produced by recombinant methods. Nucleic acids encoding the antibodies are introduced into host cells and expressed using materials and procedures described herein. In one instance, the antibodies are produced in mammalian host cells, such as CHO cells. Monoclonal (e.g., human) antibodies may be produced by the expression of recombinant DNA in host cells or by expression in hybridoma cells as described herein.

Antibodies and antibody variants (including antibody fragments) useful in the present methods may comprise: (a) a heavy chain variable region comprising a VHCDR1 having the sequence as set forth in SEQ ID NO: 1, a VHCDR2 having the sequence of SEQ ID NO: 2, a VHCDR3 having the sequence of SEQ ID NO: 3; and (b) a light chain variable region comprising a VLCDR1 having the sequence of SEQ ID NO: 5, a VLCDR2 having the sequence of SEQ ID NO: 6, and a VLCDR3 having the sequence of SEQ ID NO: 7.

Also contemplated for use in the methods disclosed herein is an anti-IL-33 antibody or antibody variant thereof comprising a heavy chain variable region (VH) domain at least 95%, 90%, or 85% identical to the sequence set forth in SEQ ID NO: 4. In some instances the anti-IL-33 antibody or antibody variant thereof comprises a light chain variable region (VL) domain at least 95%, 90%, 85% identical to the sequence set forth in SEQ ID NO: 8. In some instances, the anti-IL-33 antibody or antibody variant thereof comprises: (a) a heavy chain variable region (VH) at least 95%, 90%, or 85% identical to the sequence set forth in SEQ ID NO 4; and (b) a light chain variable region (VL) at least 95%, 90%, 85% identical to the sequence set forth in SEQ ID NO: 8.

In some instances, the anti-IL-33 antibody is 33_640087_7B, as disclosed in WO2016/156440, which is incorporated herein by reference. 33_640087_7B, also referred to in the art as MEDI3506 and tozorakimab, is an anti-IL-33 antibody that binds to the reduced form of IL-33 (redIL-33) with high affinity. 33_640087_7B also inhibits the conversion of redIL-33 to the oxidised form (oxIL-33), which has been shown to induce signalling via RAGE and induce epithelial cell proliferation.

33_640087_7B is an exemplary anti-IL-33 antibody having: (a) a heavy chain variable region comprising a VHCDR1 having the sequence as set forth in SEQ ID NO: 1, a VHCDR2 having the sequence of SEQ ID NO: 2, a VHCDR3 having the sequence of SEQ ID NO: 3; and (b) a light chain variable region comprising a VLCDR1 having the sequence of SEQ ID NO: 5, a VLCDR2 having the sequence of SEQ ID NO: 6, and a VLCDR3 having the sequence of SEQ ID NO: 7.

33_640087_7B also comprises a VH domain having the amino acid sequence as set forth in SEQ ID NO: 4 and a VL domain having the amino acid sequence as set forth in SEQ ID NO: 8.

33_640087_7B is an IgG1 antibody, the sequence of the full length light chain and heavy chain of 33_640087_7B, including the IgG1 chain, is set forth in SEQ ID NOs: 9 and 10, respectively.

In some instances the anti-IL-33 antibody or antibody variant thereof has similar, or the same pharmacokinetic (pK) characteristics as 33_670087_7B in humans.

In particular, the anti-IL-33 antibody or antibody variant may have a similar, or the same, half-life in humans as 33_670087_7B. The anti-IL-33 antibody or antibody variant having a similar, or the same, half-life in humans as 33_670087_7B, when administered at a dose of 30 mg Q2W, may have a half-life of about 10 to about 20 days, about 12 to about 15 days, or of about 12.7 days. The anti-IL-33 antibody or antibody variant having a similar, or the same, half-life in humans as 33_670087_7B, when administered at a dose of 100 mg Q2W, may have a half-life of about 10 to about 20 days, about 12 to about 15 days, or of about 13.2 days. The anti-IL-33 antibody or antibody variant having a similar, or the same, half-life in humans as 33_670087_7B, when administered at a dose of 300 mg Q2W, may have a half-life of about 10 to about 20 days, about 12 to about 15 days, or of about 14.8 days.

In some instances, the IL-33 antibody or variant thereof may competitively inhibit binding of IL-33 to 33_640087-7B (as described in WO2016/156440). WO2016/156440 discloses that 33_640087-7B binds to redIL-33 with particularly high affinity and attenuates both ST-2 and RAGE-dependent IL-33 signaling. An antibody or variant thereof is said to competitively inhibit binding of a reference antibody to a given epitope if it specifically binds to that epitope to the extent that it blocks, to some degree, binding of the reference antibody to the epitope. Competitive inhibition may be determined by any method known in the art, for example, solid phase assays such as competition ELISA assays, Dissociation-Enhanced Lanthanide Fluorescent Immunoassays (DELFIA®, Perkin Elmer), and radioligand binding assays. For example, the skilled person could determine whether an antibody or variant thereof competes for binding to IL-33 by using an in vitro competitive binding assay, such as the HTRF assay described in WO2016/156440, paragraphs 881-886, which is incorporated herein by reference. For example, the skilled person could label 33_640087-7B with a donor fluorophore and mix multiple concentrations with fixed concentration samples of acceptor fluorophore labelled-redIL-33. Subsequently, the fluorescence resonance energy transfer between the donor and acceptor fluorophore within each sample can be measured to ascertain binding characteristics. To elucidate competitive binding antibody molecules, the skilled person could first mix various concentrations of a test binding molecule with a fixed concentration of the labelled 33_640087-7B antibody. A reduction in the FRET signal when the mixture is incubated with labelled IL-33 in comparison with a labelled antibody-only positive control would indicate competitive binding to IL-33. An antibody or variant thereof may be said to competitively inhibit binding of the reference antibody to a given epitope by at least 90%, at least 80%, at least 70%, at least 60%, or at least 50%.

In various instances, the anti-IL-33 antibody or antibody variant thereof selected from human antibody, a humanized antibody, a chimeric antibody, a monoclonal antibody, a recombinant antibody, an antigen-binding antibody fragment, a single chain antibody, a monomeric antibody, a diabody, a triabody, tetrabody, a Fab fragment, an IgG1 antibody, an IgG2 antibody, an IgG3 antibody, and an IgG4 antibody. In some instances, the anti-IL-33 antibody variant is selected from the group consisting of a diabody, a triabody, a tetrabody, a Fab fragment, single domain antibody, scFv, wherein the dose is adjusted such that the binding sites to be equimolar to those dosed by bivalent antibodies.

In some instances, the anti-IL-33 antibody or antibody variant thereof binds to IL-33 comprising an amino acid sequence of SEQ ID NO:11. In various instances, the anti-IL-33 antibody or antibody variant thereof may be capable of binding to a mature form of the full-length IL-33 protein comprising an amino acid sequence of SEQ ID NO: 11. In various instances, the anti-IL-33 antibody or antibody variant thereof may be capable of binding to an IL-33 protein fragment comprising amino acids 72-270, 79-270, 95-270, 99-270, 107-270, 109-270, 111-270, or 112-270 of SEQ ID NO:11.

In various instances, the anti-IL-33 antibody or antibody variant thereof may be capable of binding to the reduced (red-IL-33) and/or the oxidised (ox-IL-33) form of IL-33. In some instances, the anti-IL-33 antibody or antibody variant thereof may be capable of preferentially binding to the reduced (red-IL-33) and/or the oxidised (ox-IL-33) form of IL-33.

In various instances, the anti-IL-33 antibody or antibody variant thereof may be an inhibitory antibody, capable of inhibiting IL-33 or a fragment thereof as defined herein. In various instances, an inhibitory antibody may be capable of inhibiting the association of IL-33 or a fragment thereof with an IL-33 receptor.

### EXAMPLES

### Example 1—Proof of Mechanism for Anti-Interleukin-33 Antibody Tozorakimab: Results of a Phase 1 Study in Healthy Adults and Participants with Chronic Obstructive Pulmonary Disease

The alarmin cytokine interleukin (IL)-33 orchestrates inflammatory and remodelling responses following tissue damage (Scott IC et al. Sci Rep 2018; 8:3363; Cohen E et al. Nat Commun 2015; 6:8327; Murdaca G et al. Int J Mol Sci 2019; 20:5856). Excess IL-33 plays a key role in initiating and driving chronic obstructive pulmonary disease in COPD (Allinne J et al. J Allergy Clin Immunol 2019; 144:1624-37.e10; Schmitz J et al. Immunity 2005; 23:479-90). Tozorakimab (MEDI3506) is a human immunoglobulin Gl monoclonal antibody that specifically and potently targets IL-33. This first-in-human study (NCT03096795) evaluated the safety, tolerability, pharmacokinetics and immunogenicity of tozorakimab. This report details proof of mechanism for tozorakimab from this study.

### Methods

The three-part, phase 1, randomized, blinded, placebo-controlled study was conducted between May 15, 2017 and Sep. 30, 2019 across two centres in the UK. In all cohorts, participants were randomized 3:1 to receive tozorakimab: placebo. This report presents data from parts 1 and 2.

Part 1 eligible participants with a history of mild atopy and sensitivity to house dust mites (HDM), received single ascending doses (SADs) of either 300 mg intravenous (IV) or 1 mg, 3 mg, 10 mg, 30 mg, 100 mg or 300 mg subcutaneous (SC) tozorakimab or placebo. Part 2 eligible participants with Global Initiative for Chronic Obstructive Lung Disease (GOLD) grade I-II COPD received multiple ascending doses (MADs) of 30 mg, 100 mg or 300 mg SC tozorakimab or placebo.

Pharmacodynamics (PD) were assessed as exploratory outcomes. Target engagement was measured with ultra-selective assays for IL-33 forms in serum (all cohorts), and in local airway nasal mucosal lining fluid (MLF) samples by non-invasive nasosorption (MAD cohort). Serum levels of sST2 were also measured. After IL-33 challenge, interferon gamma (IFN-γ) was measured ex vivo with whole blood assays (SAD cohorts). Multiplex immunoassays (Meso Scale Discovery) were used to explore the PD effects of tozorakimab on inflammatory mediators (MAD cohort). Eosinophil levels were measured in whole blood (MAD cohort).

Results Patient baseline demographics were as follows:

| | SAD cohorts | | | | MAD cohorts | | | | Japanese cohort | | | |
|---|---|---|---|---|---|---|---|---|---|---|---|---|
| | (n = 56) | | | | (n = 24) | | | | (n = 8) | | | |
| | Tozo | | Placebo | | Tozo | | Placebo | | Tozo | | Placebo | |
| | n = 42 | | n = 14 | | n = 18 | | n = 6 | | n = 6 | | n = 2 | |
| Male sex, | 41 | (97.6) | 14 | (100.0) | 10 | (55.6) | 5 | (83.3) | 6 | (100.0) | 2 | (100.0) |
| n (%) | | | | | | | | | | | | |
| Age, years, | 37.2 | (8.9) | 37.6 | (9.7) | 64.2 | (5.6) | 66.2 | (4.1) | 32.2 | (9.6) | 31.5 | (9.2) |
| mean (SD) | | | | | | | | | | | | |
| Race, n (%) | | | | | | | | | | | | |
| Asian | 6 | (14.3) | 3 | (21.4) | 0 | (0.0) | 0 | (0.0) | 6 | (100.0) | 2 | (100.0) |
| Black or | 1 | (2.4) | 1 | (7.1) | 0 | (0.0) | 0 | (0.0) | 0 | (0.0) | 0 | (0.0) |
| African | | | | | | | | | | | | |
| American | | | | | | | | | | | | |
| White | 30 | (71.4) | 8 | (57.1) | 18 | (100.0) | 6 | (100.0) | 0 | (0.0) | 0 | (0.0) |
| Other | 5 | (11.9) | 1 | (7.1) | 0 | (0.0) | 0 | (0.0) | 0 | (0.0) | 0 | (0.0) |
| Body mass | 24.7 | (2.7) | 26.9 | (2.4) | 28.0 | (4.8) | 25.6 | (4.0) | 23.8 | (2.4) | 21.0 | (1.2) |

index,
kg/m²,
mean (SD)

In total, 56 participants were enrolled and randomized in the SAD cohorts (healthy adults with mild atopy and sensitivity to HDM): 42 in the tozorakimab-treated group and 14 in the placebo-treated group. 24 patients were enrolled and randomised in the MAD cohorts (adults with GOLD grade I-II COPD): 18 in the tozorakimab-treated group and six in the placebo-treated group).

### Target Engagement Biomarker Studies (Exploratory Endpoint)

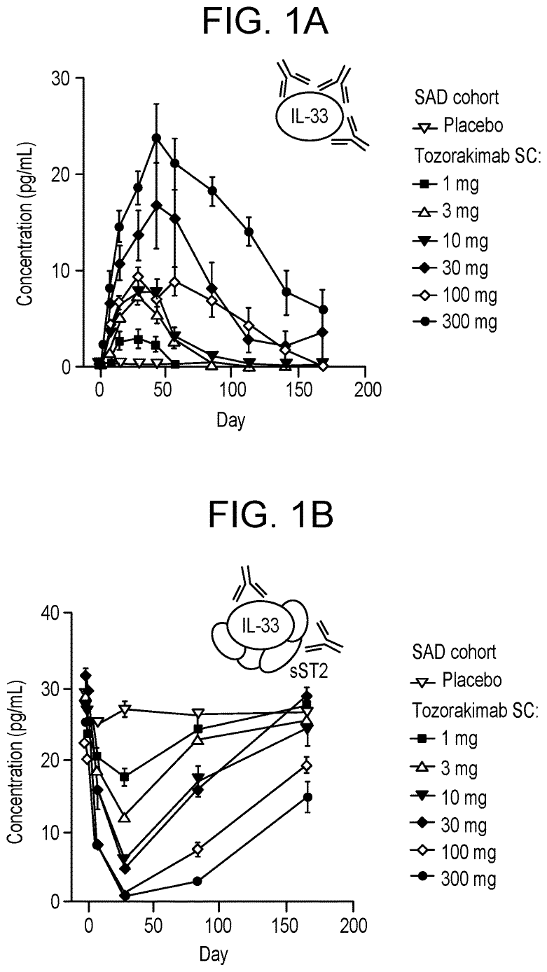

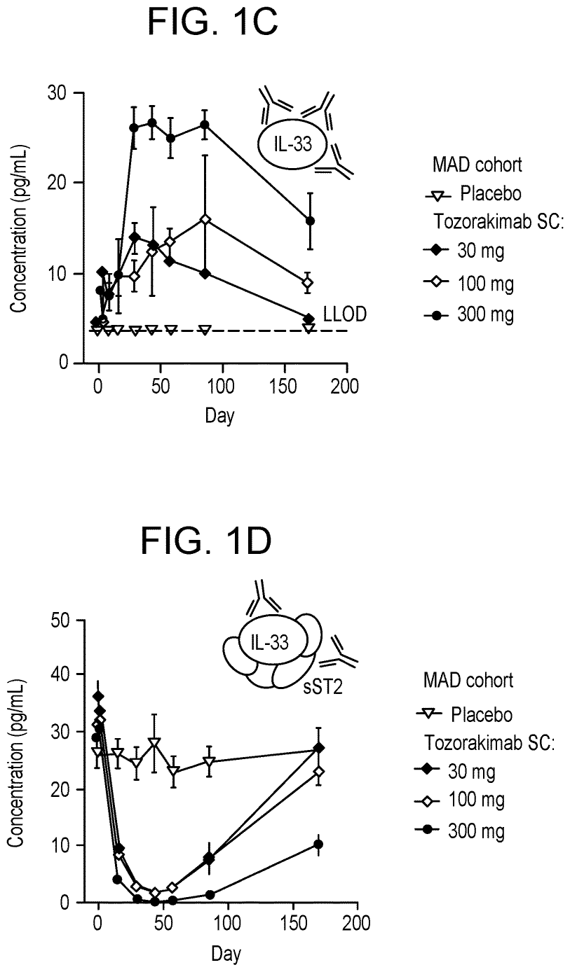

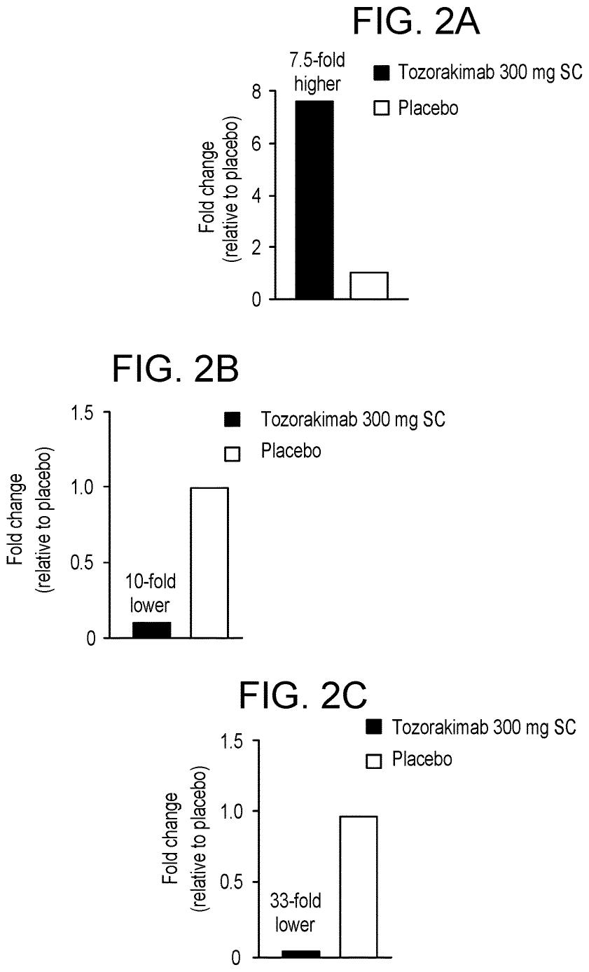

Tozorakimab target engagement was demonstrated in serum (FIG. 1) and local airway nasal MLF (FIG. 2). In serum, tozorakimab increased levels of IL-33/tozorakimab complex compared with placebo across all cohorts (FIG. 1A [SAD cohort] and 1C [MAD cohort]), whereas levels of the endogenous IL-33/sST2 complex were decreased in all cohorts (FIG. 1B [SAD cohort] and 1D [MAD cohort]). Tozorakimab did not significantly impact serum levels of total sST2 compared with placebo, at any dose level.

In local airway nasal mucosal lining fluid (MLF), tozorakimab increased levels of IL-33/tozorakimab complex compared with placebo (MAD cohort) (FIG. 2A) and decreased levels of both reduced and oxidized forms of IL-33 (FIGS. 2B and 2C).

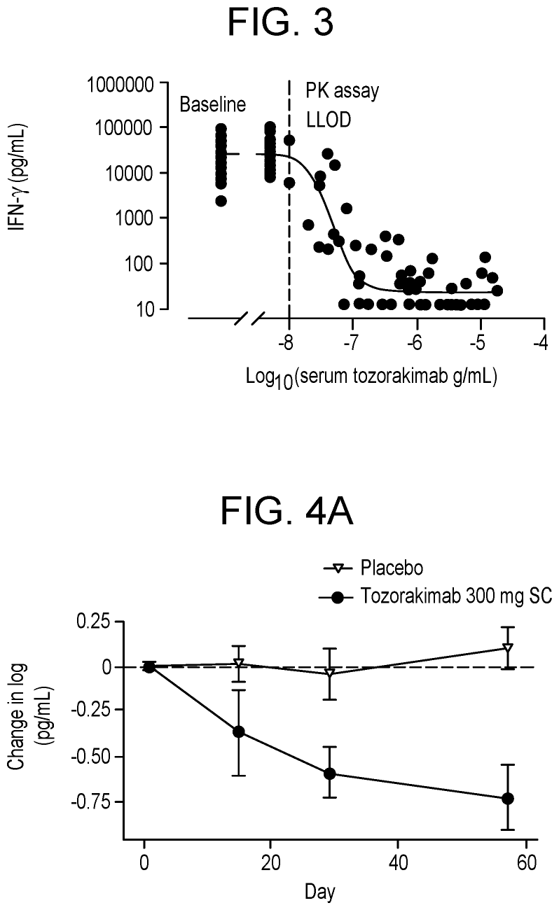

Higher levels of tozorakimab in circulation correlate with lover levels of induced IFN-γ (FIG. 3).

### Pharmacodynamic Biomarker Studies (Exploratory Endpoint)

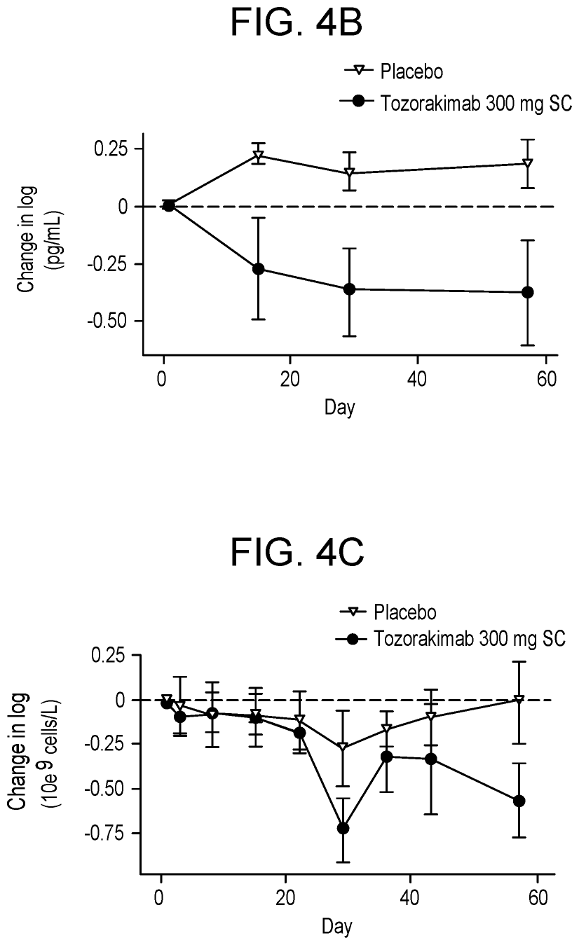

Tozorakimab (300 mg SC) significantly reduced serum IL-5 and IL-13 levels compared with placebo (FIGS. 4A and 4B). Moreover, tozorakimab significantly reduced blood eosinophil levels (FIG. 4C) and these reductions correlated with reductions in serum levels of IL-5 (repeated measures correlation [r]=0.64; 95% confidence interval [CI]: 0.23-0.86, p=0.0034) and IL-13 (r=0.75; 95% CI: 0.43-0.91, p=0.00019).

### CONCLUSIONS

These data show proof of mechanism through target engagement and identification of PD biomarkers for tozorakimab in a first-in-human study (NCT03096795) in patients with COPD. Target engagement was demonstrated in the circulation and locally in the airway using pioneering nasosorption sampling. The results support the entry of tozorakimab into phase 2 and phase 3 studies. Phase 2 (NCT04631016) and phase 3 studies (NCT05166889 and NCT05158387) are currently underway to investigate the safety and efficacy of tozorakimab for the treatment of COPD.

### Example 2—A Phase II, Randomized, Double-blind, Placebo-controlled Study to Assess the Efficacy, Safety and Tolerability of MEDI3506 in Participants with Moderate to Severe Chronic Obstructive Pulmonary Disease and Chronic Bronchitis (FRONTIER 4)

The present example describes a Phase 2 randomized, double-blinded, placebo-controlled, parallel-group, proof-of-concept study to evaluate the efficacy, safety, PK, and immunogenicity of MEDI3506 in adult subjects with moderate or severe COPD receiving Standard of Care (dual or triple therapy) as maintenance therapy. Participants also have a history of ≥1 moderate or severe acute exacerbation in the previous 12 months while on stable background treatment, and moderate to severe chronic bronchitis, with active sputum and cough symptoms

MEDI3506 (also referred to herein as 33_640087_7B) is a human IgG1 mAb that binds to human IL-33. MEDI3506 binds full length and mature forms of human IL-33 with exceptionally high affinity and prevents IL-33 binding to soluble (sST2) and membrane-bound forms of ST2 (also known as IL-1RL1) receptor.

Several clinical and non-clinical studies point to the IL-33/ST2 signalling axis playing a key role in the pathogenesis of COPD. Thus, blocking this signalling pathway could be of therapeutic benefit in COPD.

Participants must be on stable doses of dual therapy (ICS+LABA, or LABA+LAMA) or triple therapy (ICS+LABA+LAMA) for >3 months prior to enrolment and should remain so during the study. There should have been no change in maintenance COPD treatment after a previous exacerbation prior to entering into the study.

Participants will be randomised into treatment groups which will receive 600 mg MEDI3506 SC (20 mM L-histidine/L-histidine-hydrochloride, 220 mM L-arginine-hydrochloride, 0.03% (w/v) polysorbate 80, pH 5.5), or volume-matched placebo SC (referred to collectively “investigational products”); in a 1:1 ratio overall every 4 weeks (Q4W) for a total of 7 doses with the final dose at Week 24.

Participants will be enrolled in this study for at least a 4-week screening/run-in period, a 24-week intervention period (or “treatment window”) during which they receive 7 doses SC Q4W, a 4 week additional period, and an 8-week follow-up period. The investigational schema laid out in Table 2

The primary estimand is as follows: The difference in mean change from baseline in FEV1 at Week 12 (MEDI3506-placebo) will be estimated using a repeated measures mixed effects analysis of covariance model, for the ITT population. This will include all available data from all visits up to and including Week 12, irrespective of whether the participant discontinued study intervention or received reliever therapy. The model will include fixed effects for baseline, eosinophil strata, background medication strata, visit, study intervention, and the baseline by visit, and study intervention by visit interactions. An unstructured covariance matrix will be used to describe the correlations between observations on a participant between visits.

A similar approach will be taken for the analysis of cough VAS, BCSS, CASA-Q, SGRQ, and reliever medication. Data may be log-transformed prior to analysis where appropriate. Change from baseline in objective cough parameters and oscillometry parameters at Week 12 will be analyzed using analysis of covariance. Analysis of time to event and annualized rate of event data will include available data (up to Week 28 where this is available) for all participants. Time to event endpoints will be analysed

### Screening Procedures

Participants should meet the following criteria:
1. Participants must be 40 to 75 years of age inclusive.
2. Participants who are current or ex-smokers with a tobacco history of ≥10 pack-years.
3. Participants who are up-to-date on pneumococcus and influenza vaccines as per local treatment guidelines.
4. Participants who have a documented history of COPD for at least 1 year.
5. Participants who have a post-BD FEV1/FVC<0.70 and a post-BD FEV1>30% and <80% predicted normal value at screening. Centralized spirometry will be used for this criteria assessment.
6. Participants who have a physician confirmed participant history of chronic bronchitis as defined as presence of cough and sputum on most days for >3 mos/yr in at least the 2 year period immediately prior to SVI (screening).
7. Participants who have an average BCSS score of ≥2 in cough and ≥2 in sputum domains assessed over the 14 days preceding SV3.
8. Participants who have a documented stable regimen of dual therapy or triple therapy for ≥3 months prior to enrolment; there should have been no change in treatment after the previous exacerbation prior to entering into the study. Where dual therapy consists of ICS+LABA or LABA+LAMA, and triple therapy consists of ICS+LABA+LAMA. Both dual and triple therapy may be in the form of separate inhalers of fixed dose combination inhalers but may not be in nebulized form.
9. Participants who have a documented history of ≥1 moderate or severe AECOPD requiring systemic corticosteroids and/or antibiotics for at least 3 days duration (or 1 injection of depot formulation), or hospitalization for reason of AECOPD in the previous 12 months prior to screening.
10. Participants who are clinically stable and free from an exacerbation of COPD for 1 month prior to SVI (screening) and prior to Day 1.
11. Body mass index within the range 19 to 35 kg/m2 (inclusive).

### Randomisation and Administration

Randomisation will occur at study visit 3 (SV3-Day 1). Participants who continue to meet eligibility criteria will be randomised into treatment groups as described above. Blood samples, urine samples, efficacy assessments and safety assessments will be performed in order to establish baseline.

The randomization will be stratified by baseline blood eosinophils (<300 cells/μL vs≥300 cells/μL) and background medication (includes ICS vs does not include ICS).

The first investigational product (IP) administration will occur at study visit 3 (Day 1), and will comprise administering the first dose of investigational product during the treatment window. Administering 600 mg MEDI3506 will require 2× 2 mL SC injections per dose. Placebo groups will be injection volume matched to the MEDI3506 groups.

At study visit 4 (Day 2), participants will return for assessment of their adherence to self-assessment efficacy reporting procedures, and safety assessments. Procedures are outlined in Table 4. The second investigational product administration will occur at study visit 6 (Day 29±3).

The third investigational product administration will occur at study visit 7 (Day 57±3).

The fourth investigational product administration will occur at study visit 8 (Day 85±3).

The fifth investigational product administration will occur at study visit 9 (Day 113±3).

The sixth investigational product administration will occur at study visit 10 (Day 141±3).

The seventh and final investigational product administration will occur at study visit 11 (Day 169±3).

### Endpoints

The primary endpoint visit occurs at week 12, as assessed at study visit 10 (day 113±4).

The primary endpoint is improvement is change from baseline to week 12 in clinic pre-BD FEV₁. Forced expiratory volume in 1 second is a validated and clinically important endpoint in COPD studies, and has been used extensively in trials used to support registration of add on therapy to current standard of care (dual/triple therapy) in a similar chronic bronchitis patient population (Martinez et al 2015).

Based on available data, the improvement in FEV₁ assumed in the sample size determination is expected to be achieved by Week 12. However, improvement in FEV₁ is considered important but not sufficient to meet the unmet medical need in COPD. To enable evaluation of the secondary endpoint of COPDCompEx, the treatment is continued after collection of primary endpoint data, to collect further events. The longer intervention period duration also allows the exploratory assessment of treatment effect on FEV₁ beyond Week 12.

The secondary endpoint is COPDCompEx at week 28. Changes in clinic pre-BD FEV₁ will also be assessed at week 28.

Blood samples will be collected from subjects for the assessment of biomarkers that are relevant to disease pathology and/or the mechanism of action of MEDI3506.

### Results

The highest dose of MEDI3506 administered to subjects in this Phase 2 clinical study will be 600 mg by SC injection Q4W. This dose is predicted to have a lower exposure, in terms of maximum concentration at steady-state (C_{max,ss}; approximately 2.5-fold) and AUC (approximately 1.6-fold), compared with the highest dose administered (ie, single dose of 300 mg IV MEDI3506) in the Phase 1 clinical study (Study D9180C00001). A dose of 600 mg by SC injection Q4W is predicted to have a higher C_{max,ss}, but the same AUC compared with the highest multiple dose administered (ie, 300 mg SC Q2W) in the same study (Table 6).

The nature and severity of disease in the study population are not expected to significantly impact the overall exposure nor clearance. The published PK data for a monoclonal antibody licensed for use in

AD indicate that the disease status (ie, healthy subjects vs subjects with AD) had no significant impact on exposure or clearance (Kovalenko et al, 2016). Hence, we anticipate that MEDI3506 exhibits similar PK profiles for both healthy subjects and subjects with COPD.

### Example 3—Dose Selection Criteria for MEDI3506 in the Treatment of COPD

In order to select target dose a PK/PD model was generated using target engagement data from the Ph1 study (NCT03096795). More specifically, the PK/PD model was based on:
Quantitative information of MEDI3506: IL33 and IL33: ST2 complex in systemic circulation from phase 1. Both complex concentrations were measured using proprietary IL-33 detection reagents that specifically bind to the reduced form of IL-33 (redIL-33).
MEDI3506 PK data from phase 1 (linear PK, half-life (t1/2) of 17 days)

Additional preclinical information that informed dose selection includes:
redIL-33: ST2 signalling pathway
oxIL-33: RAGE: EGFR signalling pathway
   redIL-33: ST2 signalling pathway

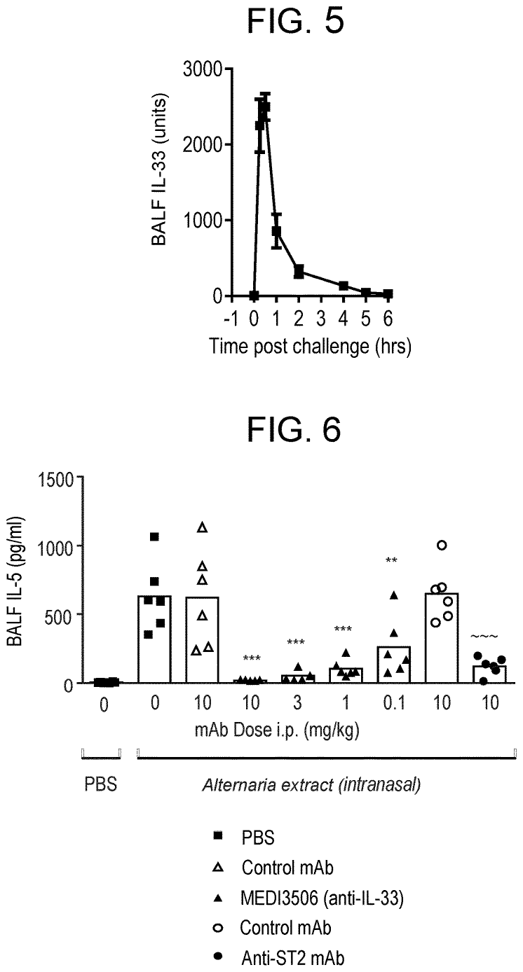

Models of Alternaria alternata (ALT) induced airway inflammation in mice have been previously described (Kouzaki et al. J. Immunol. 2011, 186:4375-4387; Bartemes et al J Immunol, 2012, 188:1503-1513). Endogenous IL-33 is released rapidly following ALT exposure and drives IL-33-dependent IL-5 production in the lung. Male or female wildtype or humanized IL-33 mice (6-10 weeks) were anaesthetized briefly with isofluorane and administered either 25 μg of ALT extract (Greer, Lenoir, NC) or vehicle intranasally in a total volume of 50 μl. Mice were treated intraperitoneally with MEDI3506 (0.1, 1, 2 or 10 mg/kg), isotype control IgG (NIP228) or vehicle (PBS, 10 ml/kg) at 24 hours prior to intranasal challenge with ALT. At 24 hours after challenge, mice were terminally anaesthetised with pentobarbital sodium prior to exsanguination and bronchoalveolar lavage fluid (BALF) collection. BALF was collected by lavage via tracheal cannula. BALF was centrifuged, cells counted (total cells by FACS (FacsCALIBER, BD)) and supernatant was analysed for cytokines by ELISA (Meso Scale Discovery, Rockville, MD). Differential cell counts (200 cells/slide) were performed on cytospin preparations stained with Diff-Quik (Fisher Scientific, UK). All work was carried out to UK Home Office ethical and husbandry standards under the authority of an appropriate project licence. A dose dependent inhibition of IL5 by MEDI3506 was observed in BALF with significant suppression achieved at the lowest dose tested in this study 0.1 mg/kg. At 3 mg/kg 90% of inhibition was achieved corresponding to a mean serum systemic exposure of 20 μg/mL in mouse. The results are shown in FIGS. 5 and 6.

oxIL-33: RAGE: EGFR signalling pathway

It has recently been discovered that the oxidised form of IL-33 (oxIL-33, IL-33ox or IL-33DSB) directly impairs epithelium repair responses, decreases epithelial goblet cell differentiation and proliferation, and increased mucus productions and production of mucin-associated genes, such as MUC5AC. It was found that oxIL-33 mediates pathological effects on the epithelium by binding to and signalling through a complex of RAGE and EGFR (as described in WO2021/089563, which is hereby incorporated by reference).

The following MEDI3506 concentrations with respect to the oxIL-33 signalling pathway were used to inform dose selection:
Threshold to reverse oxIL-33-mediated dysfunctional scratch wound closure

### Scratch Wound Closure

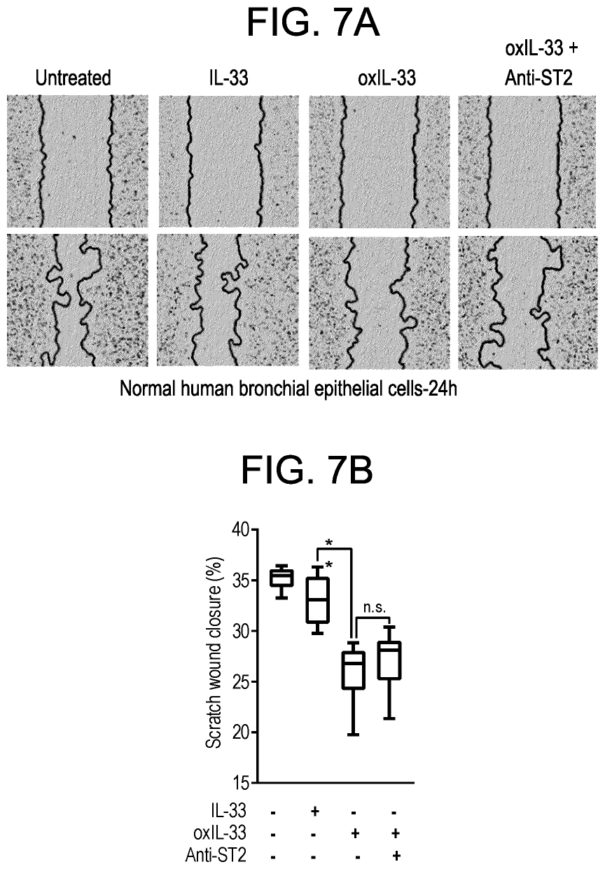

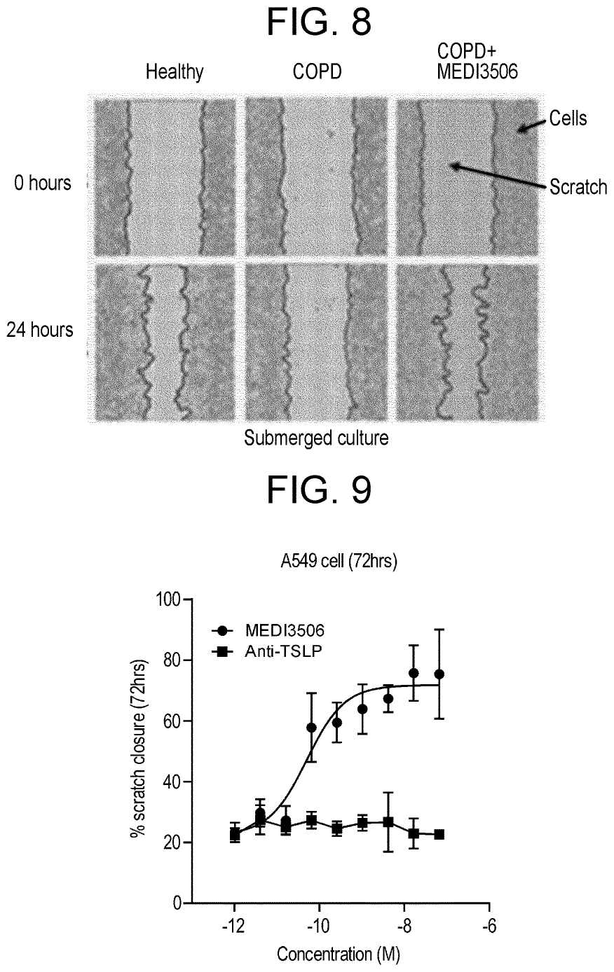

Previous experiments have shown that oxIL-33 impairs epithelial scratch wound closure in healthy human bronchial epithelial cells (FIGS. 7A and 7B). The impaired wound closure was not reversed with anti-ST2 antibody treatment, indicating that the pathological effect is mediated via the oxIL-33-RAGE/EGFR signalling axis. Scratch wound impairment was also seen in bronchial epithelial cells obtained from COPD subjects (FIG. 8).

The concentration of MEDI3506 needed to reverse oxIL-33-mediated scratch wound closure dysfunction was calculated in A549 cells cultures.

A549s were obtained from ATCC and cultured in RPMI GlutaMax medium supplemented with 1% Penicillin/Streptomycin and 10% FBS. Cells were harvested with accutase (PAA, #L1 1-007) and seeded into 96 well plates at 5×10⁵/100 μl and incubated at 37° C., 5% CO₂ for 6-8 hours. The wells were then washed twice with 100 μl of PBS before addition of 100 μl of starve media (RPMI GlutaMax medium supplemented with 1% Penicillin/Streptomycin) and incubated at 37° C., 5% CO₂ for 18-24 hours. Using a WoundMaker™ (Essen Bioscience), cells were scratched and then wells were washed 2× with 200 μl of PBS before addition of RPMI GlutaMax medium supplemented with 0.1% FBS (v/v) and 1% (v/v) Penicillin/Streptomycin containing the indicated stimulations; media alone (unstimulated control), different concentrations of MEDI3506 or anti-TSLP antibody, returned to 37° C., 5% CO₂. Plates were placed into an IncucyteZoom for wound healing imaging and analysis over a 72 hour period. Relative Wound Density was calculated through the wound healing algorithm within the Incucyte Zoom software. FIG. 9 shows the MEDI3506 dose-dependent improvement in scratch wound closure in A549 cells. Concentrations of MEDI3506 greater than 50.4 μM (or 7.26 ng/ml) were needed to achieve full response. This has been assumed to be equivalent to a concentration in the blood of 0.15 μg/mL (assuming 5% distribution to epithelial lining fluid following sub-cutaneous administration). No effect was seen for anti-TSLP antibody.

### Integrative PK/PD Model for Target Engagement

An integrated popPK/PD model was established based on SAD/MAD/IV MEDI3506 systemic exposure and target engagement (TE) clinical data from the MEDI3506 Ph1 study. TE information used were systemic IL33-MEDI3506 complex formation and IL33-ST2 reduced levels from FTIM.

Population and doses covered in the model include:
healthy subjects with mild atopy after single sub-cutaneous (SC) doses (1 to 300 mg SC) and 300 mg intravenous (IV)·
Mild COPD patients receiving multiple doses of 30, 100 and 300 mg SC

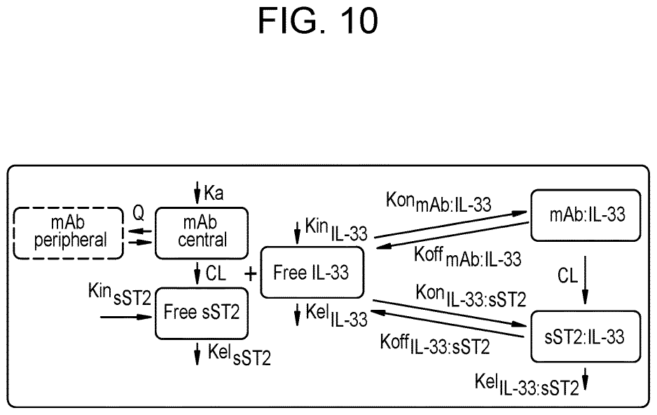

The model structure has four compartments defined, and is shown in FIG. 10.

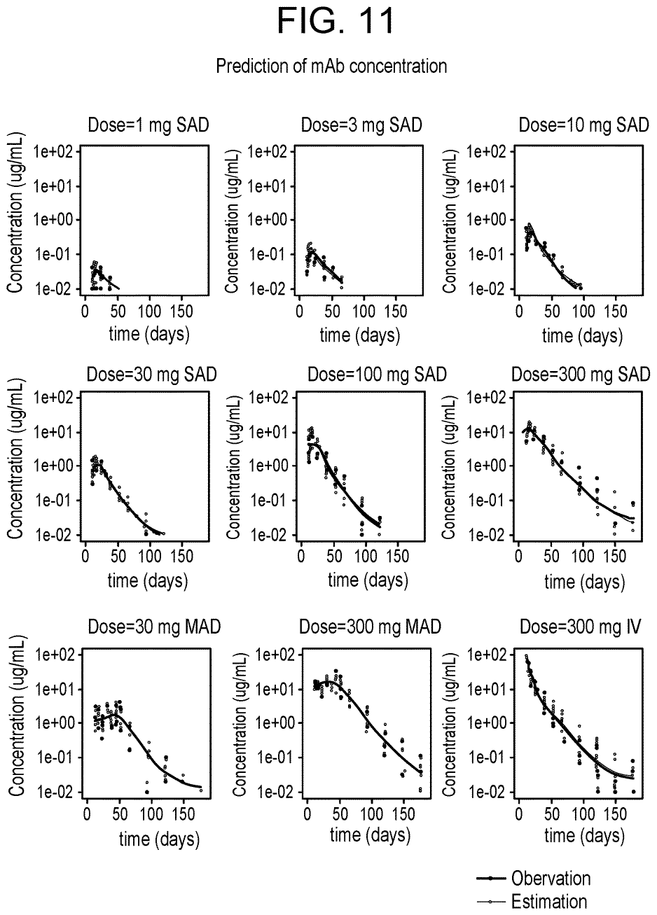

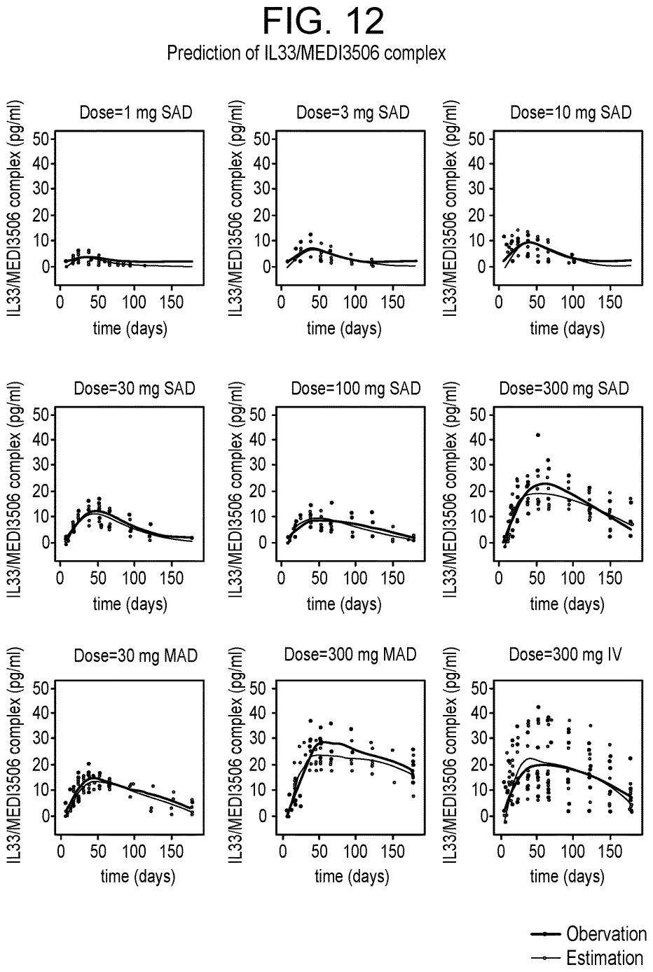

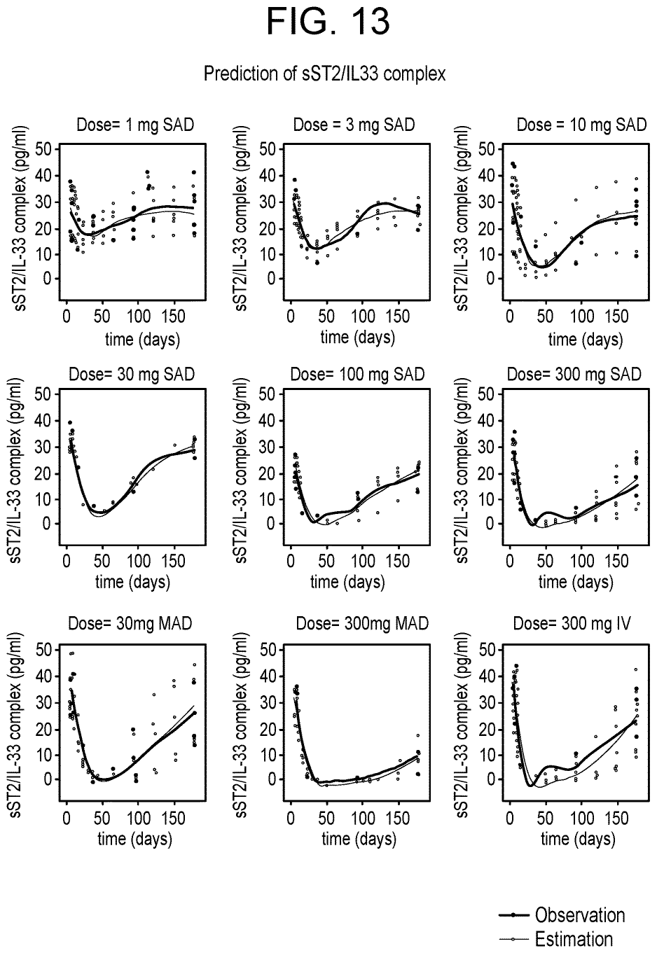

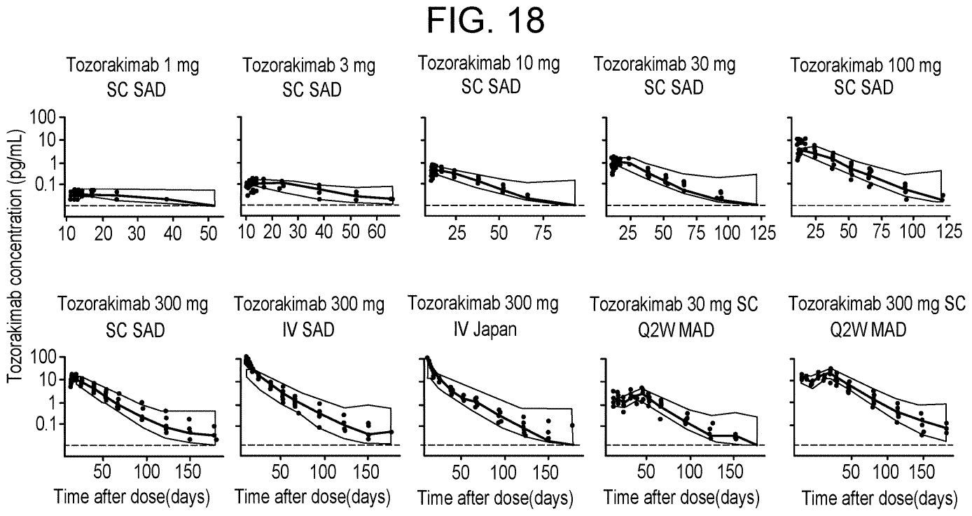

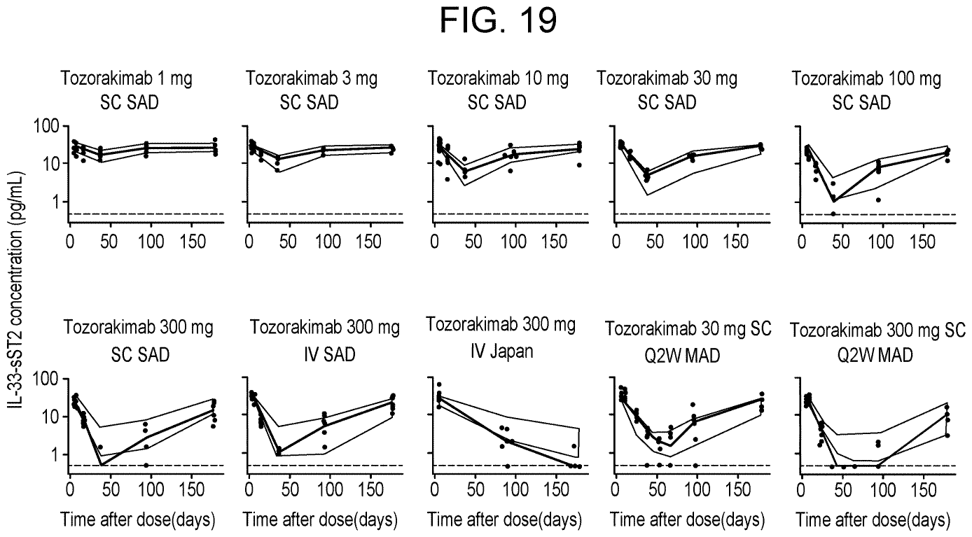

The PKPD model reliably describes observed PK profiles of MEDI3506, the MEDI3506: IL33 complex formation and IL33: ST2 dose dependent inhibition in the blood (FIGS. 11, 12 and 13). FIGS. 18 and 19 show a different presentation of FIGS. 11 and 13, respectively. The solid lines represent the medians of the observations. The shaded areas represent the 95% confidence intervals of the medians predicted by the model. The dashed lines represent the LLOQ values for tozorakimab (0.01 ng/ml) and IL-33: sST2 (0.5 μg/ml), respectively.

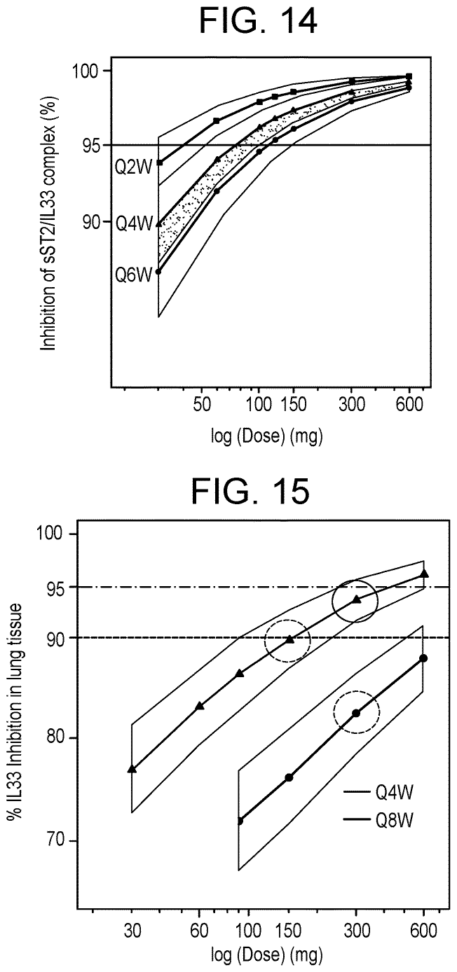

The dose response in ST2: IL33 complex inhibition for MEDI3506 in blood at trough for Q2W, Q4W and Q6W dosing regiments is shown in FIG. 14.

The PK/PD model for IL-33/sST2 complex inhibition in blood has been further transformed to predict the IL-33 inhibition in the lung tissue (assumptions: blood: tissue partition coefficient of 14%, and that IL-33 levels in the lung are two-fold higher than in blood).

The % IL-33 inhibition in lung tissue at trough for the Q4W and Q8W dosing frequencies is shown in FIG. 15.

Dose selection:

Almost 95% target inhibition in lung tissue at trough is predicted for 300 mg dose Q4W dosing frequency. MEDI3506 300 mg Q8W is predicted to achieve TE in the lung of greater than 80%, which means that sustained inhibition of IL-33 in the lungs of patients may be achievable using a longer but more convenient dosing interval for patients (FIG. 15).

Based on Ph1 PK data and other input parameters, MEDI3506 serum concentrations were modelled for 300 mg Q4W and 300 mg Q8W. Both of these regimens are predicted to have a trough concentration higher than the amount predicted from the Alternaria humanised IL-33 mouse model required to achieve 60% inhibition (FIG. 16). The Alternaria model is more representative of an acute effect caused in exacerbations or viral infections. As such, the dose projection using this cut off is likely to be sufficient to achieve an efficacious dose for human chronic IL-33-mediated diseases, such as COPD. Both regimens are also predicted to have trough concentrations higher than the threshold amount identified by the scratch wound model to inhibit pathological signalling via the oxIL-33: RAGE/EGFR signalling axis (FIG. 17).

### Example 4—A Phase III, Multicentre, Randomised, Double-Blind, Chronic-Dosing, Parallel-Group, Placebo-Controlled Study to Evaluate the Efficacy and Safety of Two Dose Regimens of MEDI3506 in Participants with Symptomatic Chronic Obstructive Pulmonary Disease (COPD) with a History of COPD Exacerbations

### Overall Design

The purpose of this Phase III study is to evaluate the efficacy and safety of MEDI3506 300 mg every 8 weeks (Q8W) and 300 mg every 4 weeks (Q4W) dose regimens administered subcutaneously (SC) in adult participants with symptomatic COPD and history of ≥2 moderate or ≥1 severe exacerbation of COPD in the previous 12 months. Participants should be receiving optimised treatment with maintenance inhaled therapy (ICS/LABA/LAMA triple therapy, or dual therapy if triple is not indicated or contraindicated) in stable doses throughout at least 3 months prior to enrolment.

The study will randomise approximately 1272 participants, stratified by region, maintenance inhaled therapy (dual vs triple), and smoking status (current vs former). The study will include former and current smokers. Participants will continue the same COPD maintenance therapy throughout the duration of the study.

The study will consist of an at least 2-week screening period, a 52-week treatment period (with the site visits and IP administrations every 4 weeks), and an 8 week post treatment follow-up period.

Key Primary and Secondary objectives and endpoints are described in the following table:

| Objective | Endpoint/Estimand |
|---|---|
| Primary | |
| To evaluate the effect of 2 dose regimens of | Endpoint: annualised rate of moderate to |
| MEDI3506 as add on to SoC compared with | severe COPD exacerbations. |
| SoC plus placebo on the rate of moderate to | Summary measure: rate ratio. |
| severe COPD exacerbations in former | Population: full analysis set, former smokers. |
| smokers. | Participants who are former smokers with |
| | symptomatic COPD and a history of |
| | exacerbations, on optimised treatment with |
| | maintenance inhaled therapy. |
| | Intercurrent events: treatment policy. All data |
| | up to Week 52 will be included regardless of |
| | IP discontinuation or changes in maintenance |
| | inhaled therapy. |
| | Supportive analysis: while-on-treatment ^{c}. |
| Key secondary | |
| To evaluate the effect of 2 dose regimens of | Endpoint: annualised rate of moderate to |
| MEDI3506 as add on to SoC compared with | severe COPD exacerbations. |
| SoC plus placebo on the rate of moderate to | Summary measure: rate ratio. |
| severe COPD exacerbation in former and | Population: full analysis set, former or current |
| current smokers. | smokers. Participants who are former smokers |
| | or current smokers with symptomatic COPD |
| | and history of exacerbations, on optimised |
| | treatment with maintenance inhaled therapy. |
| | Intercurrent events: treatment policy. |
| To evaluate the effect of 2 dose regimens of | Endpoint: time to first moderate to severe |
| MEDI3506 as add on to SoC compared with | COPD exacerbation. |
| SoC plus placebo on time to moderate to | Summary measure: hazard ratio. |
| severe COPD exacerbations. | Population: full analysis set, former smokers. |
| | Intercurrent events: treatment policy. |
| To evaluate the effect of 2 dose regimens of | US key secondary |
| MEDI3506 as add on to SoC compared with | Endpoint: change from baseline in pre-BD, |
| SoC plus placebo on change in pre-BD lung | pre-dose trough FEV₁ (mL) at Week 52. |
| function. | Summary measure: difference in mean change |
| | from baseline. |
| | Population: full analysis set, former smokers. |
| | Intercurrent events: treatment policy. |
| | Ex-US key secondary |
| | Endpoint: change from baseline in pre-BD, |
| | pre-dose trough FEV₁ (mL) over 52 weeks. |
| | Summary measure: difference in mean change |
| | from baseline. |
| | Population: full analysis set, former smokers. |
| | Intercurrent events: treatment policy. |
| To evaluate the effect of 2 dose regimens of | US key secondary |
| MEDI3506 as add on to SoC compared with | Endpoint: proportion of participants achieving |
| SoC plus placebo on respiratory symptoms. | MCID in E-RS: COPD total score (percentage |
| | of participants with a decrease in E-RS: COPD |
| | total score of ≥2 points from baseline at |
| | Week 52). |
| | Summary measure: odds ratio. |
| | Population: full analysis set, former smokers. |
| | Intercurrent events: composite. |
| | Ex-US key secondary |
| | Endpoint: change from baseline in E-RS: |
| | COPD total score over 52 weeks. |
| | Summary measure: Difference in mean change |
| | from baseline. |
| | Population: full analysis set, former smokers. |
| | Intercurrent events: treatment policy. |
| To evaluate the effect of 2 dose regimens of | US key secondary |
| MEDI3506 as add on to SoC compared with | Endpoint: proportion of participants achieving |
| SoC plus placebo on respiratory health | MCID in SGRQ score (percentage of |
| status/health-related quality of life. | participants with a decrease in SGRQ total |
| | score of ≥4 points from baseline at Week 52). |
| | Summary measure: odds ratio. |
| | Population: full analysis set, former smokers. |
| | Intercurrent events: composite. |
| | Ex-US key secondary |
| | Endpoint: change from baseline in SGRQ total |
| | score over 52 weeks. |
| | Summary measure: difference in mean change |
| | from baseline. |
| | Population: full analysis set, former smokers. |
| | Intercurrent events: treatment policy. |
| Secondary | |
| To evaluate the effect of 2 dose regimens of | Time to first severe COPD exacerbation b |
| MEDI3506 as add on to SoC compared with | Annualised rate of severe COPD |
| SoC plus placebo on severe COPD | exacerbations. |
| exacerbations. | |
| To further evaluate the effect of 2 dose | Change from baseline in CAT total score at |
| regimens of MEDI3506 as add on to SoC | Week 52. |
| compared with SoC plus placebo on COPD | Proportion of participants achieving MCID in |
| health status/health-related quality of life. | CAT score (percentage of participants |
| | with a decrease in CAT total score of ≥2 |
| | points from baseline at Week 52). |
| To evaluate the effect of 2 dose regimens of | Proportion of participants having ≥1 HRU |
| MEDI3506 as add on to SoC compared with | type. |
| SoC plus placebo on overall and | Annualised rate of hospitalisations. |
| COPD-related HRU. | Annualised rate of ER/ED visits. |
| | Annualised rate of hospitalisations and ER/ED |
| | visits combined. |
| | Number of days in hospital, measured by total |
| | number of days in hospital, general ward, |
| | ICU, CCU, and any other HRU, if there is |
| | sufficient data. |
| | Annualised rate of unscheduled |
| | visits/tests/procedures, including |
| | unscheduled visits to study sites due to |
| | COPD. |
| | Rate of hospital readmissions. |
| To evaluate the effect of 2 dose regimens of | Change from baseline (difference in mean |
| MEDI3506 as add on to SoC compared with | number of puffs/day) in rescue medication use |
| SoC plus placebo on daily rescue medication | over 52 weeks. |
| use. | |

AE = adverse event; BD = bronchodilator; CAT = COPD Assessment Test; CCU= critical care unit; COPD = chronic obstructive pulmonary disease; ECG = electrocardiogram; ED = emergency department; ER = emergency room; E-RS: COPD = Evaluating Respiratory Symptoms in COPD; FEV1 = forced expiratory volume in one second; HRU = healthcare resource utilisation; ICU = intensive care unit; IP = investigational product; MCID = minimal clinically important difference; SGRQ = St. George's Respiratory Questionnaire; SoC = standard of care.

### Type of Participants and Disease Characteristics

1 Documented diagnosis of COPD for at least one year prior to enrolment.

2 Post-BD FEV₁/FVC<0.70 and post-BD FEV₁>20% of predicted normal value (as assessed by central spirometry at screening).

3 Documented history of ≥2 moderate or ≥1 severe COPD exacerbations within 12 months prior to enrolment:
(a) An exacerbation is considered moderate if it required treatment with systemic corticosteroids and/or antibiotics, and severe if it required hospitalisation. Note: hospitalisation is defined as an inpatient admission ≥24 hours in the hospital, in an observation area, emergency department, or other equivalent healthcare facility depending on the country and healthcare system.
(b) At least one qualifying exacerbation should have been treated with systemic corticosteroids.
(c) Events treated with antibiotics alone qualify as a moderate exacerbation only when antibiotic was specifically prescribed for worsening of COPD symptoms.
(d) Previous exacerbations should be confirmed to have occurred while the participant was on stable dual or triple (ICS/LABA/LAMA) maintenance inhaled therapy for COPD and not as a result of a gap or step down in the treatment.
(e) At least one qualifying exacerbation should have occurred while on the most recent stable uninterrupted therapy prior to enrolment.

4 Documented optimised treatment with COPD maintenance therapy (ICS/LABA/LAMA triple therapy, or dual therapy if triple is not indicated or contraindicated) and at a stable dose for at least 3 months prior to enrolment.

5 Smoking history of ≥10 pack-years:
(a) Former smokers will be defined as participants who are currently not smoking and with smoking cessation ≥6 months prior to screening with an intention to quit permanently.
(b) Current smokers will be defined as participants who are currently smoking tobacco (at least one cigarette per day on average during the past 7 days) and are not currently participating in smoking cessation.
(c) Electronic cigarette (e-cigarette) use does not contribute to the pack-year count for eligibility.

6 CAT total score ≥10, with each of the phlegm (sputum) and cough items with a score ≥2, at both screening and randomisation.

7 At least 70% daily PRO completion during the entire screening period, with at least 50% daily PRO completion in the 14-day period prior to randomisation.

8 At least 70% compliance with COPD maintenance inhaled therapy (defined as taking COPD maintenance inhaled medication as scheduled for the day) during the entire screening period.

9 Able to read and use electronic devices.

### Study Intervention

### Investigational Products (IPs)

| | MEDI3506 300 mg | MEDI3506 300 mg | |
|---|---|---|---|
| Arm Name | Q8W | Q4W | Placebo |
| Intervention | MEDI3506 | MEDI3506 | Placebo |
| Name | | | |
| Type | Biologic | Biologic | Placebo |
| Dosage Form | Vial | Vial | Vial |
| Unit Dose | 150 mg/mL | 150 mg/mL | Not applicable |
| Strength(s) | | | |
| Dosage Level(s) | Dosing Q4W alternately | 300 mg Q4W | Equivalent volume to |
| | with 300 mg MEDI3506 | | MEDI3506 Q4W |
| | and placebo | | |
| Route of | SC injection | SC injection | SC injection |
| Administration | | | |
| IMP and NIMP | IMP | IMP | NIMP |
| Sourcing | Provided centrally by | Provided centrally by | Provided centrally by |
| | the sponsor | the sponsor | the sponsor |
| Packaging and | Single vial kits with a | Single vial kits with a | Single vial kits with a |
| Labelling | unique number that is | unique number that is | unique number that is |
| | printed on all labels | printed on all labels | printed on all labels |
| | within the kit (ie, the | within the kit (ie, the | within the kit (ie, the |
| | outer carton label and | outer carton label and | outer carton label and |
| | the label of the vial | the label of the vial | the label of the vial |
| | within the carton). | within the carton). | within the carton). |
| | Labels will be prepared | Labels will be prepared | Labels will be prepared |
| | in accordance with GMP | in accordance with GMP | in accordance with GMP |
| | and local regulatory | and local regulatory | and local regulatory |
| | requirements. | requirements. | requirements. |

### Rescue Medicine

Short-acting β2-agonists (SABAs, eg, salbutamol, albuterol, terbutaline, levalbuterol), short acting muscarinic antagonists (SAMA), SABA/SAMA combination or alternative rescue medication, as per local standard of care, may be used during the study in the event of worsening of COPD symptoms.

### Maintenance Therapy

Stable optimised maintenance inhaled therapy (ICS/LABA/LAMA triple therapy, or dual therapy if triple is not indicated or contraindicated) Any other COPD maintenance therapy (eg, xanthines, antibiotics, PDE4 inhibitors etc) doses and regimen should be stable for 3 months prior to the study and throughout the study period.

### Efficacy Assessments

### Assessment of COPD Exacerbations

For the purpose of the protocol, a COPD exacerbation will be defined as a worsening in the participant's usual COPD symptoms (eg, dyspnoea, sputum volume, sputum purulence, cough, wheezing, and other COPD-related symptoms and/or findings) that is beyond normal day-to-day variation, is acute in onset, lasts 2 or more days (or less if the worsening is so rapid and profound that the treating physician judges that intensification of treatment cannot be delayed), and may warrant a change in regular medication and leads to any of the following:

Use of systemic corticosteroids for at least 3 days; a single depot injectable (IM) dose of corticosteroids will be considered equivalent to a 3-day course of systemic corticosteroids.

Use of antibiotics.

An inpatient hospitalisation due to COPD (defined as an inpatient admission ≥24 hours in the hospital, an observation area, the emergency department, or other equivalent healthcare facility depending on the country and healthcare system).

Results in death.

An exacerbation will be considered moderate if it requires treatment with systemic corticosteroids and/or antibiotics and does not meet severe event criteria. An exacerbation will be considered severe if it results in hospitalisation or death due to COPD.

The start of an exacerbation is defined as the start date of systemic corticosteroids or antibiotic treatment or hospital admission, whichever occurs earlier, and the end date is defined as the last day of systemic corticosteroids or antibiotic treatment or hospital discharge, whichever occurs later. A single depot injectable dose of corticosteroids will be considered equivalent to a 3-day course of systemic corticosteroids. The corresponding stop date for this treatment will consequently be determined as the date of administration plus 2 days.

### Spirometry (Pre- and Post-bronchodilator Assessments)

All spirometry is performed pre-dose.

Lung function (FEV₁ and FVC) will be measured by spirometry using equipment provided by a central vendor. Spirometry will be performed by the investigator or authorised delegate according to American Thoracic Society (ATS)/European Respiratory Society (ERS) guidelines (Graham et al 2019).

### Spirometry References

The Global Lung Function Initiative equations will be used to determine the predicted normal value (PNV) and are pre-programmed into the spirometer (Quanjer et al 2012).

Forced expiratory volume in one second expressed as percent of the PNV, will be calculated as follows:

$${FEV}_{1} \% of PNV = ( {FEV}_{1} measured / {FEV}_{1} PNV ) \times 100$$

FEF25-75% will be calculated using a similar method to FEV₁.

### Post-BD Spirometry

Endpoint maximal BD will be induced using albuterol (90 μg metered dose) or salbutamol (100 μg metered dose) with or without a spacer device up to a maximum of 4 inhalations within 30 minutes±15 minutes of the final pre-BD spirometry measurement. Post-BD spirometry will be performed 15 to 30 minutes later. If a participant cannot tolerate 4 puffs of albuterol or salbutamol, a lower number of inhalations may be considered at the investigator's clinical judgement.

### Patient-Reported Outcomes (PRO)

Participants will complete the following non-daily PROs in the following order: SGRQ, CAT, 5-level EuroQol-5 Dimension (EQ-5D-5L), Work Productivity and Activity Impairment-General

Health (WPAI-GH), PGIS, and Patient Global Impression of Change (PGIC). Refer to the SoA (Section Error! Reference source not found.) for frequency of assessments.

Exacerbations of Chronic Pulmonary Disease Tool-Patient-reported Outcome (EXACT-PRO)

The EXACT-PRO is a 14-item PRO instrument developed to assess the frequency, severity and duration of COPD exacerbations (Jones et al 2011, Leidy et al 2011). The instrument was developed for daily, at home, administration using a handheld electronic device. Respondents are instructed to complete the diary each evening just prior to bedtime and to answer the questions while considering their experiences “today”. The daily EXACT-PRO total score has a range of 0 to 100 with higher scores indicative of greater severity. Total score changes are used to identify the onset and recovery from an EXACT-PRO defined exacerbation event. In identifying event onset and recovery, the EXACT-PRO can provide information on event frequency and duration as well as event severity.

### Evaluating Respiratory Symptoms in COPD (E-RS)

The E-RS: COPD is an 11-item PRO developed to evaluate the severity of respiratory symptoms of COPD (Leidy et al 2014a, Leidy et al 2014b). The E-RS: COPD is a subset of items from the EXACT-PRO. The E-RS: COPD was designed to be captured as part of the daily EXACT-PRO assessment. Summation of E-RS: COPD item responses produces a total score ranging from 0 to 40, with higher scores indicating greater severity. In addition to the total score, symptom domain scores can be calculated for breathlessness (5 items; score range: 0 to 17), cough and sputum (3 items; score range: 0 to 11) and chest symptoms (3 items; score range: 0 to 12) by summing the responses of items within a respective domain. As with the total score, higher domain scores indicate greater severity. Individual score decrease of at least 2 points in the E-RS: COPD total score is considered meaningful and will be used as the responder definition (Leidy et al 2014a).

### Breathlessness, Cough and Sputum Scale (BCSS)

The BCSS is a 3-item PRO (Leidy et al 2003a, Leidy et al 2003b) that assesses the severity of breathlessness, cough, and sputum on a scale of 0 to 4. Item scores are summed to yield a total score, with higher scores indicating more severe symptoms.

### St George's Respiratory Questionnaire (SGRQ)

The SGRQ is a 50-item PRO instrument developed to measure the health status of participants with airway obstruction diseases (Jones et al 1991, Jones and Forde 2009). The questionnaire is divided into 2 parts: Part 1 consists of 8 items pertaining to the severity of respiratory symptoms in the preceding 4 weeks; Part 2 consists of 42 items related to the daily activity and psychosocial impacts of the individual's respiratory condition. The SGRQ yields a total score and 3 component scores (symptoms, activity, and impacts). The total score indicates the impact of disease on overall health status. This total score is expressed as a percentage of overall impairment, in which 100 represents the worst possible health status and 0 indicates the best possible health status. Likewise, the component scores range from 0 to 100, with higher scores indicative of greater impairment. Individual score decrease of at least 4 points in the SGRQ total score is considered meaningful and will be used to support the responder definition. Specific details on the scoring algorithms are provided by the developer in a user manual (Jones and Forde 2009).

### COPD Assessment Test (CAT)

The CAT is an 8-item PRO developed to measure the impact of COPD on health status (Jones et al 2009, Kon et al 2014). The instrument uses semantic differential 6-point response scales which are defined by contrasting adjectives to capture the impact of COPD. Content includes items related to cough, phlegm, chest tightness, breathlessness going up hills/stairs, activity limitation at home, confidence leaving home, sleep, and energy. Each item response ranges from 0 to 5 with 0 having the least impact and 5 having the greatest impact on health status. The CAT total score is the sum of item responses with score range from 0 to 40 with higher scores indicative of greater COPD impact on health status. Individual score decrease of at least 2 points in the CAT total score is considered meaningful and will be used to support the responder definition (Kon et al 2014).

5-level EuroQol-5 Dimension (EQ-5D-5L)

The EQ-5D-5L is a 5-level standardised instrument for use as a measure of health outcome. Applicable to a wide range of health conditions and treatment, it provides a simple descriptive profile and a single index value for health status. The EQ-5D-5L consists of 2 assessments, a descriptive system, and a visual analogue scale (VAS). The descriptive system comprises of the following 5 dimensions: mobility, self-care, usual activities, pain/discomfort, and anxiety/depression. Each dimension has 5 severity levels: no problems, slight problems, moderate problems, severe problems, and extreme problems. The EQ-5D-5L index score can be calculated based upon participants' responses to the 5 dimensions and using an appropriate value set, which will be further described in the statistical analysis plan (SAP).

The EQ-5D VAS records the respondent's self-rated health on a 20 cm, 0 to 100 vertical scale with endpoints labelled “the best health you can imagine” and “the worst health you can imagine”, with higher scores corresponding to a better health state. This information is used as a quantitative measure of health as judged by the individual respondents.

### Work Productivity and Activity Impairment Questionnaire (WPAI-GH)

The WPAI-GH (version 2.0) is a self-administered tool comprised of 6 questions which address absenteeism, presenteeism (reduced effectiveness while working), overall work productivity loss (absenteeism plus presenteeism), and activity impairment. This validated tool captures data from the past 7 days. The WPAI-GH outcomes are scored as impairment percentages, with a higher percentage indicating greater impairment and less productivity (Reilly et al 1993).

### Patient Global Impression of Severity (PGIS)

The PGIS is a single item designed to capture the participant's perception of overall COPD symptom severity at the time of completion using a 6-point scale (0-no symptoms to 5-very severe).

### Patient Global Impression of Change (PGIC)

The PGIC is a single item designed to capture the participant's perception in change in overall COPD symptoms since first dose of IP using a 7-point scale (1-much better to 7-much worse).

### Composite Endpoint for Exacerbations of COPD (COPDCompEx)

The composite endpoint for exacerbations of COPD (COPDCompEx) is an endpoint based on combining exacerbations with daily PRO defined events and study drop out (Vogelmeier et al 2020). The definitions for COPDCompEx components are as follows: Exacerbation: episodes leading to one or more of the following: hospitalisation, emergency room visit, treatment with systemic corticosteroids, or treatment with antibiotics.

Daily PRO events: defined by threshold and slope criteria using the following PRO variables: individual items of the BCSS and rescue medication use.

### Statistical Considerations

### Primary Endpoint

The primary endpoint is the annualised rate of moderate-to-severe exacerbations. This will be assessed for each dose of MEDI3506 vs placebo first in the primary population (former smokers) and then in the overall population of current and former smokers.

Moderate-to-severe exacerbations rate in each MEDI3506 dose regimen group will be compared with moderate-to-severe exacerbation rate in the placebo group using a negative binomial model. The response variable in the model will be the number of COPD exacerbations experienced by a participant over the full double-blind 52-week treatment period. The model will include covariates of treatment group, region, maintenance inhaled therapy (triple or dual), and the number of exacerbations in the previous year (1 vs ≥2) as categorical factors, and post-BD FEV₁% predicted at screening and log screening blood eosinophil count as continuous covariates. The logarithm of the participant's corresponding follow-up time will be used as an offset variable in the model. For the analysis in the overall population, smoking status will also be included as a covariate.

The estimated treatment effect (ie, the rate ratio of each dose of MEDI3506 versus placebo), corresponding 95% confidence interval (CI), and 2-sided p-value for the rate ratio will be presented. In addition, the model adjusted exacerbation rate in each treatment group will be presented.

A course of treatment with systemic corticosteroids or antibiotics started within 7 days of finishing the previous treatment course will be considered as treatments for the same single exacerbation.

### Secondary Endpoints

Analysis for all secondary endpoints will be conducted in the primary population (former smokers). A similar analysis will be conducted in the overall population (former and current smokers).

### Time to First Moderate or Severe COPD Exacerbations

Time to first moderate or severe COPD exacerbation will be analysed as a key secondary efficacy variable to the primary objective to explore the extent to which treatment with each dose of MEDI3506 delays the time to first exacerbation compared with placebo. A Cox proportional hazard model will be fitted with the covariates of treatment group, region, maintenance inhaled therapy, the number of exacerbations in the previous year, post-BD FEV₁% predicted at screening, and log screening blood eosinophil count. Hazard ratios, 95% CI and p-values will be reported as well as the proportion of participants with an event.

### St George's Respiratory Questionnaire

Change from baseline in SGRQ total score over 52 weeks will be compared between MEDI3506 and placebo using a repeated measures linear model. The dependent variable will be the change from baseline in SGRQ total score at post-baseline protocol-specified visits up to the Week 52 visit. Treatment, visit, treatment-by-visit interaction, region, maintenance inhaled therapy and the number of exacerbations in the previous year will be fitted as categorical covariates with baseline SGRQ total score, post-BD FEV₁% predicted, and log screening blood eosinophil count as continuous covariates. An unstructured variance covariance matrix will be used to model correlation within a participant. Contrasts will be used to produce treatment effect estimates at each visit (including at Week 24 and Week 52) and over 52 weeks. This will be reported alongside the 2-sided 95% CI and p-value.

Responder analyses will be performed for SGRQ total score at Week 52. Responders are defined as participants with a ≥4.0 points improvement (decrease) over baseline. Participants who discontinue from the study for any reason or have missing data at Week 52 will be classified as non responders. Logistic regression will be used to compare the treatment groups with treatment, region, maintenance inhaled therapy, and the number of exacerbations in the previous year as categorical covariates and post-BD FEV₁% predicted, log screening blood eosinophil count, and baseline SGRQ total score as continuous covariates. P-values and odds ratios with 95% CI will be produced for each treatment comparison.

Change from Baseline in E-RS: COPD Total Score

Change from baseline in E-RS: COPD total score over 52 weeks will be analysed using a similar model to change from baseline in SGRQ total score. Responder analyses for E RS: COPD total score at Week 52 based upon a ≥2 point improvement (decrease) from baseline will also be produced similarly to SGRQ responder analysis.

Change from Baseline in Pre-dose FEV₁

Change from baseline in pre-dose/pre-BD FEV₁ will be analysed using a similar repeated measures analysis model as for change from baseline in SGRQ score, but with treatment, visit, treatment-by-visit interaction, region, maintenance inhaled therapy and the number of exacerbations in the previous year fitted as categorical covariates, and baseline FEV₁ and log screening blood eosinophil count as continuous covariates. Contrasts will be used to produce treatment effect estimates at each visit (including at Week 24 and Week 52) and over 52 weeks. This will be reported alongside the 2-sided 95% CI and p-value.

### Other Secondary Endpoints

Time to first severe exacerbation and annualised rate of severe exacerbations will be analysed in a similar manner to moderate or severe exacerbations as described above.

Analyses of change from baseline in CAT total score and the proportion of participants with ≥2 point decrease (improvement) in CAT total score will be conducted using similar methods as SGRQ total score.

### Example 5—A Phase II, Randomized, Double-blind, Placebo-controlled Study to Assess the Efficacy, Safety and Tolerability of MEDI3506 in Participants with Uncontrolled Moderate-to-Severe Asthma

The present example describes a Phase II, randomised, double-blind, placebo-controlled, parallel-group, proof-of-concept study to evaluate the efficacy, safety, pharmacokinetics (PK) and immunogenicity of MEDI3506 in adult participants with uncontrolled moderate-to-severe asthma on standard of care (SOC). Participants will be randomised in a 1:1: 1 ratio to receive 600 mg MEDI3506, 300 mg MEDI3506, or placebo every 4 weeks (Q4W) by subcutaneous (SC) injection for a total of 4 doses. Participants will be enrolled in this study for up to 29 weeks. The study comprises of 3 periods including the screening period of up to 5 weeks, an intervention period of 16 weeks, and a follow-up period of 8 weeks.

MEDI3506 (also referred to herein as 33_640087_7B) is a human IgG1 mAb that binds to human IL-33. MEDI3506 binds full length and mature forms of human IL-33 with exceptionally high affinity and prevents IL-33 binding to soluble (sST2) and membrane-bound forms of ST2 (also known as IL-1RL1) receptor.

Several clinical and non-clinical studies point to the IL-33/ST2 signalling axis playing a key role in the pathogenesis of asthma. Thus, blocking this signalling pathway could be of therapeutic benefit in asthma.

The MEDI3506 pre-clinical profile suggests that MEDI3506 can reduce asthmatic airway inflammation, improve epithelial integrity, reduce mucus production, and improve muco-ciliary transport. As such, MEDI3506 is hypothesised to impact on asthma disease status by increasing FEV₁ (and other physiological measures of lung function) and reducing frequency and severity of asthma exacerbations, thereby improving quality of life.

### Screening Procedures

Participants will be assessed for study eligibility at study visit 1 (SV1-between days-35 to −28). Participants assessed to have uncontrolled asthma at SV1 will undergo further baseline assessments at SV1. Participants will then record adherence to their SOC asthma controller and reliever medication for a minimum period of 14 days before returning for SV2 (between days −14 to −6). At SV2, participants whose asthma remains uncontrolled despite acceptable adherence to asthma controller medication (≥70% adherence [days] to controller medication documented in eDiary and ACQ-6 ≥1.5) will undergo further screening assessments. Participants who continue to meet all eligibility criteria will be asked to attend SV4 (randomisation) 6 to 14 days following SV2.

Baseline measurements of ACQ-6, SGRQ, FeNO, pre-BD and post-BD spirometric assessments will be collected throughout the screening period.

### Endpoints and Assessments

The primary efficacy endpoint is the effect of treatment compared to placebo on lung function, as measured in clinic, by a change from baseline to week 16, in pre-BD FEV₁ (L).

Secondary endpoints include the assessment of the effect of treatment compared to placebo on asthma control, as measured by a change from baseline to week 16 in ACQ-6 score. This includes measurement of the proportion of subjects with a decrease in ACQ-6 score of greater than or equal to 0.5 from baseline to week 16. This also includes measurement of the proportion of subjects achieving ACQ-6 well controlled status (defined as ACQ-6 score of less than or equal to 0.75 at week 16).

The assessment also includes monitoring the effect of treatment compared to placebo on lung function, as measured in clinic, by a change from baseline to weeks 8 and 16, in post-BD FEV₁ (L).

Secondary endpoints also include the assessment of the effect of treatment compared with placebo on CompEx (CompEx annualized event rate) based on the period from baseline to week 16.

Secondary endpoints also include the assessment of the effect of treatment compared with placebo on concentration of FeNO, as measured by a percent change from baseline to week 16 in concentration of FeNO in exhaled breath.

Further endpoints for assessing longitudinal effect of treatment compared with placed on asthma control and health status include:
Change from baseline to Weeks 1, 4, 8, 12, and 24 in ACQ-6 score.
Change from baseline to Weeks 4, 12, and 24 in SGRQ score.
Change from baseline up to Week 24 in 2-weekly mean daily rescue medication usage (puffs/day).
Change from baseline to Weeks 4, 8, 12, 16, 20 and 24 in:
   Asthma symptom score.
   Mean number of night-time awakenings.

Further endpoints for assessing the effect of treatment compared with placebo on asthma-related inflammatory airway biomarkers include percent change from baseline to Weeks 1, 4, 8, 12, 16, 20, and 24 in concentration of FeNO.

Key Primary and Secondary objectives and endpoints are summarised in the following table:

| Objective | Endpoint |
|---|---|
| Primary | |
| To assess the effect of MEDI3506 | As measured in clinic, change from baseline to |
| compared with placebo on lung function, in | Week 16 in pre-BD FEV1 (L). |
| adult participants with uncontrolled | |
| moderate-to-severe asthma. | |
| Secondary | |
| To further assess the effect of MEDI3506 | As measured in clinic, change from baseline to |
| compared with placebo on lung function, in | Weeks 8 and 16 in post-BD FEV1 (L). |
| adult participants with uncontrolled | |
| moderate-to-severe asthma. | |
| To assess the PK of MEDI3506 in adult | Serum MEDI3506 concentration-time profiles |
| participants with uncontrolled moderate-to- | during the intervention and follow-up periods. |
| severe asthma. | |
| To assess the immunogenicity of | ADA during the intervention and follow-up |
| MEDI3506 in adult participants with | periods. |
| uncontrolled moderate-to-severe asthma. | |
| To assess the effect of MEDI3506 | Change from baseline to Week 16 in ACQ-6 score. |
| compared with placebo on asthma control | Proportion of participants with a decrease in |
| in adult participants with uncontrolled | ACQ-6 score of ≥0.5 from baseline to Week 16. |
| moderate-to-severe asthma. | Proportion of participants achieving ACQ-6 well |
| | controlled status (defined as ACQ-6 score ≤ 0.75 |
| | at Week 16). |
| To assess the effect of MEDI3506 | Change from baseline to Week 16 in SGRQ score. |
| compared with placebo on health status in | Proportion of participants with a decrease in SGRQ |
| adult participants with uncontrolled | total score of ≥4 points from baseline to Week 16. |
| moderate-to-severe asthma. | |
| To assess the effect of MEDI3506 | Time to first CompEx event based on the period |
| compared with placebo on CompEx in adult | from baseline to Week 16. |
| participants with uncontrolled moderate-to- | CompEx annualised event rate. |
| severe asthma. | |
| To assess the effect of MEDI3506 | Percent change from baseline to Week 16 in |
| compared with placebo on concentration of | concentration of FeNO in exhaled breath. |
| FeNO in adult participants with | |
| uncontrolled moderate-to-severe asthma. | |

### Study Intervention

Investigational Products (IPs)

| Intervention | | |
|---|---|---|
| Name | MEDI3506 | Placebo |
| Type | Biologic | Drug |
| Dose | Supplied in 2R vial with nominal 1 mL of | Supplied in 3-cc vial with nominal 1 |
| Formulation | MEDI3506 containing 20 mM L-histidine/ | mL solution of 20 mM L-histidine/ |
| | L-histidine-hydrochloride, 220 mM | L-histidine-hydrochloride, 240 mM |
| | L-arginine-hydrochloride, 0.03% (w/v) | sucrose, 0.02% (w/v) polysorbate 80, |
| | polysorbate 80, pH 5.5 | pH 6.0 |
| Unit Dose | 150 mg/mL | Not applicable |
| Strength | | |
| Dosage Level | 600 mg Q4W and 300 mg Q4W | 4 mL Q4W |
| Route of | SC injection | SC injection |
| Administration | | |

Rescue Medicine Systemic corticosteroid therapy may be used in the case of asthma exacerbation during the study. Although the use of rescue medications is allowable at any time during the study, the use of rescue medications should be delayed, if possible, for at least 2 hours following the administration of study intervention. The date and time of rescue medication administration as well as the name and dosage regimen of the rescue medication must be recorded.

### Efficacy Assessments

### Spirometry (Pre- and Post-bronchodilation)

The Global Lung Function Initiative equations will be used to determine the PNV and are pre-programmed into the spirometer. FEV1, expressed as percent of the PNV, will be calculated as follows:

$$FEV 1 \% of PNV = ( FEV 1 measured / FEV 1 PNV ) \times 100$$

Bronchodilatation can be induced using albuterol (90 μg metered dose), salbutamol (100 μg metered dose) or levalbuterol (45 μg metered dose) up to a maximum of 4 inhalations. It is highly recommended to use a spacer device for this procedure.

After a gentle and complete exhalation, up to a maximum of 4 inhalations of salbutamol (100 μg metered dose) or albuterol (90 μg metered dose) should be administered using a spacer device. In rare cases where a participant has an adverse or allergic reaction to albuterol/salbutamol, levalbuterol (45 μg metered dose, up to a maximum of 4 inhalations) can be used (Sorkness et al 2008). A nebuliser should not be used. A lower total dose (eg, 2 inhalations instead of 4 and if required up to a maximum of 4 puffs) can be used if there is a concern about any effect on the participant's safety; the reason should be noted in the participant's medical record.

The highest technically acceptable pre- and post-BD FEVIwill be used to determine reversibility. Reversibility is calculated as follows:

$$FEV 1 \% Reversibility = ( post - BD FEV 1 - pre - BD FEV 1 ) / pre - BD FEV 1 \times 100$$

### Fractional Exhaled Nitric Oxide

Airway inflammation will be evaluated using a standardised single-breath FeNO test. A single exhalation technique recommended by the manufacturer will be followed (Allakhverdi et al 2007; Alving et al 2017). The FeNO test will be performed prior to AO and spirometry. Participants should follow the relevant medication and other restrictions beforehand (Sections 6.5.3 and 5.3). If any of the above restrictions are not met, and the assessment cannot be sufficiently delayed on the day the assessment should be rescheduled within the allowed visit window.

The NIOX VEROR Airway Inflammation Monitor will be used to measure FeNO. Instructions for use of this monitor will be provided in a separate user's manual. NIOX VERO® sensors will be replaced as recommended by the manufacturer. The vendor supplying the equipment will be responsible for ensuring that the equipment and procedures for the measurement of FeNO are validated prior to the start of the study.

If possible, all post-randomisation FeNO assessments should be performed within +1.5 hours of the time that the randomisation FeNO was performed.

### Assessment and Documentation of Asthma Exacerbations

During the study, an asthma exacerbation will be defined as a change in the participant's usual asthma symptoms that leads to any of the following:
a. A temporary bolus/burst of systemic corticosteroids (or a temporary increase in stable OCS background dose) for at least 3 consecutive days to treat symptoms of asthma worsening; a single depo-injectable dose of corticosteroids will be considered equivalent to a 3-day bolus/burst of systemic corticosteroids.
b. An emergency room or urgent care visit (defined as evaluation and treatment for <24 hours in an emergency department or urgent care centre) due to asthma that required systemic corticosteroids (as per the above).
c. An in-patient hospitalisation (defined as admission to an inpatient facility and/or evaluation and treatment in a healthcare facility for >24 hours).

A hospitalised asthma exacerbation is defined as any worsening of asthma that leads to (c.) above. Note: for each exacerbation, the criterion/criteria met to confirm exacerbation status should be documented.

The list below defines what is acceptable documentation for historical exacerbations:
Discharge summaries from a hospital, emergency room, or an urgent care facility indicating that a participant was hospitalised/treated with systemic corticosteroids for an asthma exacerbation.
Signed and dated notes from a referring physician, including information regarding diagnosis and treatment of an exacerbation with systemic corticosteroids.
Evidence of prescriptions for systemic corticosteroids used during an exacerbation.
A documented conversation between the Investigator (or delegate) and a participant who is already on an OCS action plan, including the information necessary to assess Inclusion Criterion 19.
A documented conversation between the treating/referral physician or nurse/nurse practitioner certifying that a participant was treated for an exacerbation with corticosteroids at their clinic or under their supervision. The dates (month/year) of the exacerbations and verbal confirmation that appropriate prescriptions were provided is necessary. This option should be used only if reasonable attempts to procure participant records have been unsuccessful.

The guidance below defines assessment of asthma exacerbations.

The start of an exacerbation is defined as the earliest of the following:
Start date of systemic corticosteroids or temporary increase in a stable OCS background dose.
Date of emergency room or urgent care visits requiring systemic corticosteroids.
Date of hospital admission due to asthma.

The end date of an exacerbation is defined as the latest of the following:
Last date of systemic corticosteroids or temporary increase in a stable OCS background dose.
Date of discharge from emergency room or urgent care visit.
Date of hospital discharge.

If less than 7 days have elapsed since the end date of an asthma exacerbation and the start date of a new asthma exacerbation, the second event will be considered a relapse of the prior asthma exacerbation in the statistical analysis.

All asthma exacerbations that occur during the treatment period and follow-up, must be recorded in the exacerbation eCRF.

Any sustained increase in asthma symptoms as reported via the eDiary (that do not meet the definition of criteria of an asthma exacerbation) will be graded as a worsening of asthma. Specifically:
An increase in rescue medication use of 4 or more puffs on at least 2 consecutive days compared with the average use during baseline or use of 12 puffs/day on any one day, and/or
n additional nebulised B2 agonist use on at least 2 consecutive days compared with the average use during baseline, and/or
An increase of 2 or more nights with awakenings due to asthma requiring rescue medication over a 7-day period compared with the average during baseline, and/or
≥6 out of previous 7 nights with awakenings due to asthma requiring rescue medication

Alerts to study site will be triggered for participants who experience a drop in PEF ≥30% on 2 consecutive days.

If an asthma exacerbation event is not associated with at least one of the above eDiary data deterioration criteria, the investigator will have to justify the decision for defining the event as an exacerbation and record it in the eCRF. Events that are not supported by any objective assessment will be deemed not to be a protocol-defined exacerbation.

### CompEx

CompEx is a combination of exacerbations of asthma and diary events (ie, combination of eDiary variables). CompEx is a composite surrogate endpoint for exacerbations of asthma, recently developed by AstraZeneca (it is not yet a regulatory-approved clinical endpoint). Diary events are defined by the threshold and slope criteria using the following morning/evening (AM/PM) diary variables:
PEF
Symptom score (0-3)
Use of rescue medication

CompEx can predict treatment efficacy on exacerbations in early development before running traditional long-term exacerbation trials. CompEx can be used broadly in the assessment of new therapeutic interventions for asthma (Fuhlbrigge et al 2017; note: the referenced publication used the term ‘severe exacerbation’ but used the same definition as used for ‘exacerbation’ in this protocol; therefore, the term ‘exacerbation’ has been used in this section for internal consistency of the protocol.)

### Participant Reported Outcome Questionnaires

Participants will complete the PRO questionnaires on an electronic device supplied to the site. PRO questionnaires to be completed at the site visits must be completed prior to treatment administration and ideally before any discussions of health status or other study procedures, such as collection of laboratory samples to avoid biasing the participant's responses to the questions. All within-window PRO assessments for the morning and evening assessments at home should be completed within this set time window programmed into the device and will notify the participant when it is time to respond to the questions.

Daily eDiary

During the study period, participants will be required to take their asthma controller therapy regularly and complete an eDiary twice daily.

At SV1, participants will receive a handheld eDiary device to complete twice-daily, daily and non-daily PRO assessments during the study. Participants will be provided training on the use of the handheld device. Daily assessments will include night-time and daytime asthma symptoms (morning and evening diary, respectively), use of inhaled rescue medication in response to worsening symptoms, nights with awakenings due to asthma symptoms (morning diary only) and background medication use. The PEF data (obtained from the home peak flow meter) will be captured at the conclusion of the morning and evening eDiary entry.

Daytime is defined as the time period between the morning lung function assessment (upon rising in the morning) and the evening lung function assessment. Night-time is defined as the time period between the evening lung function assessment (at bedtime) and the morning lung function assessment. The number of doses of rescue medication (1 dose unit=1 puff on inhaler) taken will be recorded by the participant in the eDiary twice daily. The number of inhalations taken between the morning and evening lung function assessments will be recorded in the evening.

The number of inhalations taken between the evening and morning lung function assessments will be recorded in the morning.

Nocturnal awakenings due to asthma symptoms will be recorded by the participant in the daily eDiary each morning by answering the question whether he/she woke up during the night due to asthma symptoms by a “yes” or “no” response.

Background (inhaled ICS/LABA) medication administration use will be recorded once daily in the daily eDiary as “yes” or “no” response.

### Asthma Control Questionnaire-6

The ACQ (Juniper et al 1999) was developed to measure asthma control and has been fully validated for use in adults and children 6 to 17 years of age. International guidelines for the treatment of asthma have identified that the primary clinical goal of asthma management is to optimise asthma control (minimisation of symptoms, activity limitation, bronchoconstriction, and rescue BD use) and thus reduce the risk of life-threatening exacerbations and long-term morbidity. The ACQ was developed to meet these criteria by measuring both the adequacy of asthma control and change in asthma control, which occur either spontaneously or as a result of treatment.

In the ACQ-6, participants are asked to recall how their asthma has been during the previous week by responding to one BD use question and 5 symptom questions. Questions are weighted equally and scored from 0 (totally controlled) to 6 (severely uncontrolled). The mean ACQ-6 score is the mean of the responses. Mean scores of ≤0.75 indicate well-controlled asthma, scores between 0.75 and ≤1.5 indicate partly controlled asthma, and scores>1.5 indicate not well-controlled asthma (Juniper et al 2006). Individual changes of at least 0.5 are considered clinically meaningful.

### St George's Respiratory Questionnaire

The SGRQ is a 50-item PRO instrument developed to measure the health status of patients with airway obstruction diseases (Jones et al 1991). The questionnaire is divided into two parts: part one consists of 8 items pertaining to the severity of respiratory symptoms in the preceding 4 weeks; part 2 consists of 42 items related to the daily activity and psychosocial impacts of the individual's respiratory condition. The SGRQ yields a total score and three domain scores (symptoms, activity, and impacts). The total score indicates the impact of disease on overall health status. This total score is expressed as a percentage of overall impairment, in which 100 represents the worst possible health status and 0 indicates the best possible health status. Likewise, the domain scores range from 0 to 100, with higher scores indicative of greater impairment. Specific details on the scoring algorithms are provided by the developer in a user manual (Jones and Forde 2009).

### Statistical Considerations

### Primary Endpoint

The difference in mean change from baseline in FEVlat Week 16 (MEDI3506-placebo) will be estimated using a repeated measures mixed effects analysis of covariance model, for the ITT population. The model will include all available data from all visits up to and including Week 16, irrespective of whether the participant discontinued study intervention or received rescue therapy. No imputation will be made for missing data, as a repeated measures model is being applied. The model will include fixed effects for baseline, visit, treatment and the baseline by visit and treatment by visit interactions. The significance of the treatment effect will be tested at a 10% one-sided level of significance.

### Secondary and Exploratory Endpoints

Change from baseline in ACQ-6 and SGRQ parameters will be analysed using a repeated measures analysis of covariance model, similar to that described for the primary efficacy analysis. PRO responder endpoints at Week 16 will be analysed using a chi-squared test.

Time to first CompEx event and time to first asthma exacerbation will be analysed using a Cox proportional hazard model, with treatment fitted as a covariate. The data will also be displayed in a Kaplan-Meier plot. The CompEx event rate will be analysed using negative binomial regression, with the log (follow-up time) included as an offset term. The dependent variable will be the number of CompEx events through Week 16, and the model will include treatment group as a fixed effect. All available data from participants through to Week 16 will be included, irrespective of whether they discontinued study intervention.

Change from baseline to Week 16 in concentration of FeNO will be analysed using a repeated measures analysis of covariance model, similar to that described for the primary efficacy analysis. Where appropriate, endpoints may be log-transformed prior to analysis. Estimates of the least square mean change from baseline for each treatment, and the difference between them, together with 80% confidence interval and one-sided p-values, will be obtained from the model for each visit.

The difference in mean change from baseline in post-BD FEV₁, and the change in FEV₁% reversibility at Weeks 8 and 16 will be estimated using a repeated measures mixed effects analysis of covariance model, similar to that described for the primary efficacy analysis.

The composite estimand will be the difference between MEDI3506 and placebo response rate at Week 16 in the ITT population. Participants with missing data at Week 16 will be considered non-responders. For each MEDI3506 group compared with placebo, the odds ratio and 80%, and one-sided p-values will be reported.

### REFERENCES

A number of publications are cited above in order to more fully describe and disclose the invention and the state of the art to which the invention pertains. Full citations for these references are provided below. The entirety of each of these references is incorporated herein.
Adeloye D, Chua S, Lee C, Basquill C, Papana A, Theodoratou E et al. Global and regional estimates of COPD prevalence: Systematic review and meta-analysis. J Glob Health. 2015; 5 (2): 020415.
Allakhverdi Z, Comeau M R, Jessup H K, Yoon B R, Brewer A, Chartier S et al. Thymic stromal lymphopoietin is released by human epithelial cells in response to microbes, trauma, or inflammation and potently activates mast cells. J Exp Med. 2007; 204 (2): 253-8.
Allinne J, Scott G, Lim W K, Birchard D, Erjefält J S, Sandén C et al. IL-33 blockade affects mediators of persistence and exacerbation in a model of chronic airway inflammation. J Allergy Clin Immunol. 2019; 144 (6): 1624-37. e10.
Alving K A R, Crater G, LaForce C F, Rickard K. Validation of a new portable exhaled nitric oxide analyzer, NIOX VERO: randomized studies in asthma. Pulm Ther. 2017; 3 (1): 207-18.
Bønnelykke, Klaus et al. “A genome-wide association study identifies CDHR3 as a susceptibility locus for early childhood asthma with severe exacerbations.” Nature genetics vol. 46,1 (2014): 51-5.
Byers D E, Alexander-Brett J, Patel A C, Agapov E, Dang-Vu G, Jin X et al. Long-term IL-33-producing epithelial progenitor cells in chronic obstructive lung disease. J Clin Invest. 2013; 123 (9): 3967-82.
Celli B R, MacNee W, Force AET. Standards for the diagnosis and treatment of patients with COPD: a summary of the ATS/ERS position paper. Eur Respir J. 2004; 23 (6): 932-46.
Donaldson G C, Seemungal T A, Bhowmik A, Wedzicha J A. Relationship between exacerbation frequency and lung function decline in chronic obstructive pulmonary disease. Thorax. 2002; 57 (10): 847-52.
EMA. Guideline on Clinical Investigation of Medicinal Products in the Treatment of Chronic Obstructive Pulmonary Disease 2012 [16 Jul. 2021]. Available from: https://www.ema.europa.eu/en/documents/scientific-guideline/guideline-clinical-investigation-medicinal-products-treatment-chronic-obstructive-pulmonary-disease_en.pdf Accessed on: 16 Jul. 2021.
Fuhlbrigge A L, Bengtsson T, Peterson S, Jauhiainen A, Eriksson G, Da Silva C A et al. A novel endpoint for exacerbations in asthma to accelerate clinical development: a post-hoc analysis of randomised controlled trials. The Lancet Respiratory medicine. 2017; 5 (7): 577-90.
GOLD. Global strategy for the diagnosis, management, and prevention of chronic obstructive pulmonary disease. 2020 Report 2020 [4 Aug. 2020]. Available from: https://goldcopd.org/wp-content/uploads/2019/12/GOLD-2020-FINAL-ver1.2-03Dec19_WMV.pdf. Accessed on: 4 Aug. 2020
Graham B L, Steenbruggen I, Miller M R, Barjaktarevic I Z, Cooper B G, Hall G L et al. Standardization of Spirometry 2019 Update. An Official American Thoracic Society and European Respiratory Society Technical Statement. Am J Respir Crit Care Med. 2019; 200 (8): e70-e88.
Guarascio A J, Ray S M, Finch C K, Self T H. The clinical and economic burden of chronic obstructive pulmonary disease in the USA. Clinicoecon Outcomes Res. 2013; 5235-45.
Gudbjartsson, Daniel F et al. “Sequence variants affecting eosinophil numbers associate with asthma and myocardial infarction.” Nature genetics vol. 41,3 (2009): 342-7.
Hirota, Tomomitsu et al. “Genome-wide association study identifies three new susceptibility loci for adult asthma in the Japanese population.” Nature genetics vol. 43,9 893-6. 31 Jul. 2011.
Hochberg Y. A sharper Bonferroni procedure for multiple tests of significance. Biometrika. 1988;75 (4): 800-2.
Hopkins C, Gillett S, Slack R, Lund V J, Browne J P. Psychometric validity of the 22-item Sinonasal Outcome Test. Clin Otolaryngol. 2009; 34 (5): 447-54.
Jones P W, Quirk F H, Baveystock C M. The St George's Respiratory Questionnaire. Respir Med. 1991;85 Suppl B25-31; discussion 3-7.

Jones P W, Forde Y. St George's Respiratory Questionnaire Manual. 2009; Version 2.3. Jones P W, Harding G, Berry P, Wiklund I, Chen W H, Kline Leidy N. Development and first validation of the COPD Assessment Test. Eur Respir J. 2009; 34 (3): 648-54.
Jones P W, Chen W H, Wilcox T K, Sethi S, Leidy N K, Group E-P S. Characterizing and quantifying the symptomatic features of COPD exacerbations. Chest. 2011; 139 (6): 1388-94.
Juniper E F, O'Byrne P M, Guyatt G H, Ferrie P J, King D R. Development and validation of a questionnaire to measure asthma control. Eur Respir J. 1999;14 (4): 902-7.
Juniper E F, Bousquet J, Abetz L, Bateman E D, Committee G. Identifying ‘well-controlled’ and ‘not well-controlled’ asthma using the Asthma Control Questionnaire. Respir Med. 2006; 100 (4): 616-21.
Kearley J, Silver J S, Sanden C, Liu Z, Berlin A A, White N et al. Cigarette smoke silences innate lymphoid cell function and facilitates an exacerbated type I interleukin-33-dependent response to infection. Immunity. 2015; 42 (3): 566-79.
Kemp S F, Lockey R F, Simons F E. World Allergy Organization ad hoc Committee on Epinephrine in A. Epinephrine: the drug of choice for anaphylaxis. A statement of the World Allergy Organization. Allergy. 2008; 63 (8): 1061-70.
Kim V, Crapo J, Zhao H, Jones P W, Silverman E K, Comellas A et al. Comparison between an alternative and the classic definition of chronic bronchitis in COPDGene. Ann Am Thorac Soc. 2015; 12 (3): 332-9.
Kim V, Zhao H, Regan E, Han M K, Make B J, Crapo J D et al. The St. George's Respiratory Questionnaire Definition of Chronic Bronchitis May Be a Better Predictor of COPD Exacerbations Compared With the Classic Definition. Chest. 2019; 156 (4): 685-95.
Kon S S, Canavan J L, Jones S E, Nolan C M, Clark A L, Dickson M J et al. Minimum clinically important difference for the COPD Assessment Test: a prospective analysis. Lancet Respir Med. 2014; 2 (3): 195-203.
Lahousse L, Seys LJM, Joos G F, Franco O H, Stricker B H, Brusselle G G. Epidemiology and impact of chronic bronchitis in chronic obstructive pulmonary disease. Eur Respir J. 2017; 50 (2).
Leidy N K, Rennard S I, Schmier J, Jones M K, Goldman M. The breathlessness, cough, and sputum scale: the development of empirically based guidelines for interpretation. Chest. 2003a; 124 (6): 2182-91.
Leidy N K, Schmier J K, Jones M K, Lloyd J, Rocchiccioli K. Evaluating symptoms in chronic obstructive pulmonary disease: validation of the Breathlessness, Cough and Sputum Scale. Respir Med. 2003b;97 Suppl AS59-70.
Leidy N K, Wilcox T K, Jones P W, Roberts L, Powers J H, Sethi S et al. Standardizing measurement of chronic obstructive pulmonary disease exacerbations. Reliability and validity of a patient-reported diary. Am J Respir Crit Care Med. 2011; 183 (3): 323-9.
Leidy N K, Murray L T, Monz B U, Nelsen L, Goldman M, Jones P W et al. Measuring respiratory symptoms of COPD: performance of the EXACT-Respiratory Symptoms Tool (E-R S) in three clinical trials. Respir Res. 2014a; 15124.
Leidy N K, Sexton C C, Jones P W, Notte S M, Monz B U, Nelsen L et al. Measuring respiratory symptoms in clinical trials of COPD: reliability and validity of a daily diary. Thorax. 2014b;69 (5): 443-9.
Leuppi J D, Schuetz P, Bingisser R, Bodmer M, Briel M, Drescher T et al. Short-term vs conventional glucocorticoid therapy in acute exacerbations of chronic obstructive pulmonary disease: the REDUCE randomized clinical trial. JAMA. 2013; 309 (21): 2223-31.
Mannino D M, Braman S. The epidemiology and economics of chronic obstructive pulmonary disease. Proc Am Thorac Soc. 2007; 4 (7): 502-6.
Miravitlles M, Ribera A. Understanding the impact of symptoms on the burden of COPD. Respir Res. 2017; 18 (1): 67.
Moffatt, Miriam F et al. “A large-scale, consortium-based genomewide association study of asthma.” The New England journal of medicine vol. 363,13 (2010): 1211-1221.
Müllerová H, Maskell J, Meeraus W H, Galkin D, Albers F C, Gait C. Characterization of COPD Patients Treated with Inhaled Triple Therapy Containing Inhaled Corticosteroids [ICS], Long-Acting Beta2-Agonists [LABA], and Long-Acting Muscarinic Antagonists [LAMA] in the U K. Am J Respir Crit Care Med. 2017; 195: A4986.
Mütze T, Glimm E, Schmidli H, Friede T. Group sequential designs with robust semiparametric recurrent event models. Stat Methods Med Res. 2019; 28 (8): 2385-403.
Okajima Y, Come C E, Nardelli P, Sonavane S K, Yen A, Nath H P et al. Luminal Plugging on Chest C T Scan: Association With Lung Function, Quality of Life, and COPD Clinical Phenotypes. Chest. 2020; 158 (1): 121-30.
Parikh R H, Seliger S L, Christenson R, Gottdiener J S, Psaty B M, deFilippi C R. Soluble ST2 for Prediction of Heart Failure and Cardiovascular Death in an Elderly, Community-Dwelling Population. J Am Heart Assoc. 2016; 5 (8).
Piccirillo J F, Merritt M G, Jr., Richards M L. Psychometric and clinimetric validity of the 20-Item Sino-Nasal Outcome Test (SNOT-20). Otolaryngol Head Neck Surg. 2002; 126 (1): 41-7.
Quanjer P H, Stanojevic S, Cole T J, Baur X, Hall G L, Culver B H et al. Multi-ethnic reference values for spirometry for the 3-95-yr age range: the global lung function 2012 equations. Eur Respir J. 2012; 40 (6): 1324-43.
Rabe K F, Martinez F J, Ferguson G T, Wang C, Singh D, Wedzicha J A et al. Triple Inhaled Therapy at Two Glucocorticoid Doses in Moderate-to-Very-Severe COPD. N Engl J Med. 2020; 383 (1): 35-48.
Rabe K F, Celli B R, Wechsler M E, Abdulai R M, Luo X, Boomsma M M et al. Safety and efficacy of itepekimab in patients with moderate-to-severe COPD: a genetic association study and randomised, double-blind, phase 2a trial. Lancet Respir Med. 2021.
Radicioni G, Ceppe A, Ford A A, Alexis N E, Barr R G, Bleecker E R et al. Airway mucin MUC5A C and MUC5B concentrations and the initiation and progression of chronic obstructive pulmonary disease: an analysis of the SPIROMICS cohort. Lancet Respir Med. 2021.
Reilly M C, Zbrozek A S, Dukes E M. The validity and reproducibility of a work productivity and activity impairment instrument. Pharmacoeconomics. 1993;4 (5): 353-65.
Ritchie A I, Wedzicha J A. Definition, Causes, Pathogenesis, and Consequences of Chronic Obstructive Pulmonary Disease Exacerbations. Clin Chest Med. 2020; 41 (3): 421-38.
Sampson H A, Munoz-Furlong A, Campbell R L, Adkinson N F, Jr., Bock S A, Branum A et al. Second symposium on the definition and management of anaphylaxis: summary report—Second National Institute of Allergy and Infectious Disease/Food Allergy and Anaphylaxis Network symposium. J Allergy Clin Immunol. 2006; 117 (2): 391-7.
Schmitz J, Owyang A, Oldham E, Song Y, Murphy E, McClanahan T K et al. IL-33, an interleukin-1-like cytokine that signals via the IL-1 receptor-related protein ST2 and induces T helper type 2-associated cytokines. Immunity. 2005; 23 (5): 479-90.
Seemungal T A, Donaldson G C, Paul E A, Bestall J C, Jeffries D J, Wedzicha J A. Effect of exacerbation on quality of life in patients with chronic obstructive pulmonary disease. Am J Respir Crit Care Med. 1998;157 (5 Pt 1): 1418-22.
Shrine, Nick et al. “Moderate-to-severe asthma in individuals of European ancestry: a genome-wide association study.” The Lancet. Respiratory medicine vol. 7,1 (2019): 20-34.
Smith D, Helgason H, Sulem P, Bjornsdottir U S, Lim A C, Sveinbjornsson G et al. A rare IL33 loss-of-function mutation reduces blood eosinophil counts and protects from asthma. PLOS Genet. 2017; 13 (3): e1006659.
Smith J, Owen E, Jones A, Dodd M, Webb A, Woodcock A. Objective measurement of cough during pulmonary exacerbations in adults with cystic fibrosis. Thorax. 2006; 61 (5): 425-9.
Soler-Cataluna J J, Martinez-Garcia M A, Roman Sanchez P, Salcedo E, Navarro M, Ochando R. Severe acute exacerbations and mortality in patients with chronic obstructive pulmonary disease. Thorax. 2005; 60 (11): 925-31.
Sorkness R L, Bleecker E R, Busse W W, Calhoun W J, Castro M, Chung K F et al. Lung function in adults with stable but severe asthma: air trapping and incomplete reversal of obstruction with bronchodilation. J Appl Physiol (1985). 2008; 104 (2): 394-403.
Stanczak M A, Sanin D E, Apostolova P, Nerz G, Lampaki D, Hofmann M et al. IL-33 expression in response to SARS-COV-2 correlates with seropositivity in COVID-19 convalescent individuals. Nat Commun. 2021; 12 (1): 2133.
Stone A. The application of bespoke spending functions in group-sequential designs and the effect of delayed treatment switching in survival trials. Pharm Stat. 2010; 9 (2): 151-61.
Stott-Miller M, Mullerova H, Miller B, Tabberer M, El Baou C, Keeley T et al. Defining Chronic Mucus Hypersecretion Using the CAT in the SPIROMICS Cohort. Int J Chron Obstruct Pulmon Dis. 2020; 152467-76.
Torgerson, Dara G et al. “Meta-analysis of genome-wide association studies of asthma in ethnically diverse North American populations.” Nature genetics vol. 43,9 887-92. 31 Jul. 2011.
Toy E L, Gallagher K F, Stanley E L, Swensen A R, Duh M S. The economic impact of exacerbations of chronic obstructive pulmonary disease and exacerbation definition: a review. COPD. 2010; 7 (3): 214-28.
van Manen J G, Bindels P J, Dekker F W, Bottema B J, van der Zee J S, Ijzermans C J et al. The influence of COPD on health-related quality of life independent of the influence of comorbidity. J clin epidemiol. 2003; 56 (12): 1177-84.
Vestbo J, Papi A, Corradi M, Blazhko V, Montagna I, Francisco C et al. Single inhaler extrafine triple therapy versus long-acting muscarinic antagonist therapy for chronic obstructive pulmonary disease (TRINITY): a double-blind, parallel group, randomised controlled trial. Lancet. 2017; 389 (10082): 1919-29.
Vogelmeier C F, Fuhlbrigge A, Jauhiainen A, Scheepers L, Bengtsson T, Peterson S et al. COPDCompEx: A novel composite endpoint for COPD exacerbations to enable faster clinical development. Respir Med. 2020; 173106175.
Wan, Y I et al. “Genome-wide association study to identify genetic determinants of severe asthma.” Thorax vol. 67,9 (2012): 762-8. doi: 10.1136/thoraxjnl-2011-201262
Wedzicha J A, Seemungal T A. COPD exacerbations: defining their cause and prevention. Lancet. 2007; 370 (9589): 786-96.
Corhay J L, Vincken W, Schlesser M, Bossuyt P and Imschoot J. Chronic bronchitis in COPD patients is associated with increased risk of exacerbations: a cross-sectional multicentre study. Int J Clin Pract 2013; 67 (12): 1294-301.
Crawford B, Monz B, Hohlfeld J, Roche N, Rubin B, Magnussen H, et al. Development and validation of a cough and sputum assessment questionnaire. Respir Med2008; 102 (11): 1545-55.
de Oca M M, Halbert R J, Lopez M V, Perez-Padilla R, Tálamo C, Moreno D, et al. The chronic bronchitis phenotype in subjects with and without COPD: the PLATINO study. Eur Respir J 2012; 40 (1): 28-36.
Jones P W and Forde Y. St George's respiratory questionnaire manual. 2009. Version 2.3.
Kim V, Han M K, Vance G B, Make B J, Newell J D, Hokanson J E, et al. The chronic bronchitic phenotype of COPD: an analysis of the COPDGene Study. Chest 2011; 140 (3): 626-33.
Kim V and Criner G J. Chronic bronchitis and chronic obstructive pulmonary disease. Am J Respir Crit Care Med 2013; 187 (3): 228-37.
Martinez F J, Calverley P M, Goehring U-M, Brose M, Fabbri L M, Rabe K F. Effect of roflumilast on exacerbations in patients with severe chronic obstructive pulmonary disease uncontrolled by combination multicentre controlled trial. Lancet therapy (REACT): a randomised 2015; 358 (9971): 857-66.
Monz B U, Sachs P, McDonald J, Crawford B, Nivens M C and Tetzlaff K. Responsiveness of the cough and sputum assessment questionnaire in exacerbations of COPD and chronic bronchitis. Respir Med 2010; 104 (4): 534-41.
NIH. Guidelines for the prevention and treatment of opportunistic infections in adults and adolescents with HIV. Available from URL: https://aidsinfo.nih.gov/contentfiles/lvguidelines/adult_oi.pdf. Published 2019. Accessed 17mar.2020.
Smith J, Owen E, Earis J and Woodcock A. Cough in COPD: correlation of objective monitoring with cough challenge and subjective assessments. Chest 2006; 130 (2): 379-85.
Woodruff P G, Barr R G, Bleecker E, Christenson S A, Couper D, Curtis J L, et al. Clinical Significance of Symptoms in Smokers with Preserved Pulmonary Function. N Engl J Med 2016; 374 (19): 1811-21.
For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

Sequences

| SEQ ID | | |
|---|---|---|
| NO: | Description | Sequence |
| 1. | Tozorakimab VHCDR1 | SYAMS |
| | (amino acid) | |
| 2. | Tozorakimab VHCDR2 | GISAIDQSTYYADSVKG |
| | (amino acid) | |
| 3. | Tozorakimab VHCDR3 | QKFMQLWGGGLRYPFGY |
| | (amino acid) | |
| 4. | Tozorakimab VH Domain | EVQLLESGGGLVQPGGSLRLSCAASGFTFSS |
| | (amino acid) | YAMSWVRQAPGKGLEWVSGISAIDQSTYY |
| | | ADSVKGRFTISRDNSKNTLYLQMNSLRAED |
| | | TAVYYCARQKFMQLWGGGLRYPFGYWGQ |
| | | GTMVTVSS |
| 5. | Tozorakimab VLCDR1 | SGEGMGDKYAA |
| | (amino acid) | |
| 6. | Tozorakimab VLCDR2 | RDTKRPS |
| | (amino acid) | |
| 7 | Tozorakimab VLCDR3 | GVIQDNTGV |
| | (amino acid) | |
| 8. | Tozorakimab VL Domain | SYVLTQPPSVSVSPGQTASITCSGEGMGDK |
| | (amino acid) | YAAWYQQKPGQSPVLVIYRDTKRPSGIPER |
| | | FSGSNSGNTATLTISGTQAMDEADYYCGVI |
| | | QDNTGVFGGGTKLTVL |
| 9. | Tozorakimab light chain | SYVLTQPPSVSVSPGQTASITCSGEGMGDK |
| | (amino acid) | YAAWYQQKPGQSPVLVIYRDTKRPSGIPER |
| | | FSGSNSGNTATLTISGTQAMDEADYYCGVI |
| | | QDNTGVFGGGTKLTVLGQPKAAPSVTLFPP |
| | | SSEELQANKATLVCLISDFYPGAVTVAWKA |
| | | DSSPVKAGVETTTPSKQSNNKYAASSYLSL |
| | | TPEQWKSHRSYSCQVTHEGSTVEKTVAPTE |
| | | CS |
| 10. | Tozorakimab heavy chain | EVQLLESGGGLVQPGGSLRLSCAASGFTFSS |
| | (amino acid) | YAMSWVRQAPGKGLEWVSGISAIDQSTYY |
| | | ADSVKGRFTISRDNSKNTLYLQMNSLRAED |
| | | TAVYYCARQKFMQLWGGGLRYPFGYWGQ |
| | | GTMVTVSSASTKGPSVFPLAPSSKSTSGGTA |
| | | ALGCLVKDYFPEPVTVSWNSGALTSGVHTF |
| | | PAVLQSSGLYSLSSVVTVPSSSLGTQTYICN |
| | | VNHKPSNTKVDKRVEPKSCDKTHTCPPCPA |
| | | PELLGGPSVFLFPPKPKDTLMISRTPEVTCV |
| | | VVDVSHEDPEVKFNWYVDGVEVHNAKTK |
| | | PREEQYNSTYRVVSVLTVLHQDWLNGKEY |
| | | KCKVSNKALPAPIEKTISKAKGQPREPQVYT |
| | | LPPSREEMTKNQVSLTCLVKGFYPSDIAVE |
| | | WESNGQPENNYKTTPPVLDSDGSFFLYSKL |
| | | TVDKSRWQQGNVFSCSVMHEALHNHYTQ |
| | | KSLSLSPGK |
| 11. | IL-33 full length protein | MKPKMKYSTNKISTAKWKNTASKALCFKL |
| | | GKSQQKAKEVCPMYFMKLRSGLMIKKEAC |
| | | YFRRETTKRPSLKTGRKHKRHLVLAACQQ |
| | | QSTVECFAFGISGVQKYTRALHDSSITGISPI |
| | | TEYLASLSTYNDQSITFALEDESYEIYVEDL |
| | | KKDEKKDKVLLSYYESQHPSNESGDGVDG |
| | | KMLMVTLSPTKDFWLHANNKEHSVELHKC |
| | | EKPLPDQAFFVLHNMHSNCVSFECKTDPGV |
| | | FIGVKDNHLALIKVDSSENLCT |
| | | ENILFKLSET |

1. A method of treating asthma in a subject comprising administering a therapeutically effective amount of an anti-IL-33 antibody or antibody variant thereof in a dose of from about 300 to about 600 mg at an interval of every 2 weeks (Q2W), every 4 weeks (Q4W) or 8 weeks (Q8W), wherein the anti-IL-33 antibody comprises:
a. a heavy chain variable region comprising a VHCDR1 having the sequence as set forth in SEQ ID NO: 1, a VHCDR2 having the sequence of SEQ ID NO: 2, a VHCDR3 having the sequence of SEQ ID NO: 3; and
b. a light chain variable region comprising a VLCDR1 having the sequence of SEQ ID NO: 5, a VLCDR2 having the sequence of SEQ ID NO: 6, and a VLCDR3 having the sequence of SEQ ID NO: 7.

2. A method of treating asthma in a subject comprising administering a therapeutically effective amount of an anti-IL-33 antibody or antibody variant thereof in a dose effective to achieve at least 80% inhibition of IL-33 in the lung or epithelial lining fluid (ELF), wherein the anti-IL-33 antibody comprises:
a. a heavy chain variable region comprising a VHCDR1 having the sequence as set forth in SEQ ID NO:1, a VHCDR2 having the sequence of SEQ ID NO: 2, a VHCDR3 having the sequence of SEQ ID NO: 3; and
b. a light chain variable region comprising a VLCDR1 having the sequence of SEQ ID NO: 5, a VLCDR2 having the sequence of SEQ ID NO: 6, and a VLCDR3 having the sequence of SEQ ID NO: 7.

3. The method according to claim 2, wherein the dose is effective to achieve at least about 90%, optionally at least 95%, inhibition of IL-33 in the lung.

4. The method according to claim 2 or 3, wherein the dose is about 300 to about 600 mg at an interval of every 2 weeks (Q2W), every 4 weeks (Q4W) or 8 weeks (Q8W).

5. The method according to any preceding claim, wherein the dose is about 300 mg Q8W.

6. The method according to any one of claims 1 to 4, wherein the dose is about 300 mg Q4W.

7. The method according to any one of claims 1 to 4, wherein the dose is about 600 mg Q4W.

8. The method according to any one of claims 1 to 4, wherein the dose is about 300 mg Q2W.

9. The method of any preceding claim, wherein the asthma is severe asthma.

10. The method of any of claims 1 to 8, wherein the asthma is moderate asthma.

11. The method of any of claims 1 to 8, wherein the asthma is moderate-to-severe asthma.

12. The method of claim 11, wherein the moderate-to-severe asthma is uncontrolled on standard of care.

13. The method of claim 12, wherein the standard of care is an asthma controller medication, such as medium-to-high dose inhaled corticosteroids (ICS), defined as a total daily dose of>250 μg fluticasone dry powder or equivalent, long-acting muscarinic antagonists (LAMA), leukotriene receptor antagonists (LTRA), theophylline, or oral corticosteroids.

14. The method of any preceding claim, wherein the subject is an adult.

15. The method of any preceding claim, wherein the subject is a child or an adolescent.

16. The method of any preceding claim, wherein the administration results in an increase from baseline to week 16 in pre-bronchodilator FEV₁.

17. The method of any preceding claim, wherein the administration results in a decrease from baseline to week 16 in ACQ-6 score.

18. The method of claim 17, wherein the decrease from baseline to week 16 in ACQ-6 score is greater than or equal to 0.5.

19. The method of claim 17, wherein the patient achieves ACQ-6 well controlled status at week 16.

20. The method of any preceding claim, wherein the administration results in a decrease from baseline to week 16 in SGRQ score.

21. The method of claim 20, wherein the decrease from baseline to week 16 in SGRQ score is greater than or equal to 4 points.

22. The method of any preceding claim, wherein the administration results in a decrease from baseline to week 16 in concentration of FENO in exhaled breath.

23. The method of any preceding claim, wherein the dose is effective to achieve a C_{max.ss} of from about 10 to 35 μg/ml during the dosing period.

24. The method of any preceding claim, wherein the anti-IL-33 antibody or antibody variant thereof is selected from: a human antibody, a humanized antibody, a chimeric antibody, a monoclonal antibody, a recombinant antibody, an antigen-binding antibody fragment, a single chain antibody, a monomeric antibody, a diabody, a triabody, tetrabody, a Fab fragment, an IgG1 antibody, an IgG2 antibody, an IgG3 antibody, and an IgG4 antibody.

25. The method of any preceding claim, wherein the anti-IL-33 antibody or antibody variant thereof is an IgG1.

26. The method of any preceding claim, wherein the anti-IL-33 antibody or antibody variant thereof is a human antibody.

27. The method of any preceding claim, wherein the anti-IL-33 antibody or antibody variant thereof comprises a VH domain at least 95%, 90% or 85% identical to the sequence set forth in SEQ ID NO: 4 and a VL domain at least 95%, 90% or 85% identical to the sequence set forth in SEQ ID NO: 8.

28. The method of any preceding claim, wherein the anti-IL-33 antibody comprises a VH domain sequence as set forth in SEQ ID NO:4 and a VL domain sequence as set forth in SEQ ID NO:8.

29. The method of any preceding claim, wherein the anti-IL-33 antibody comprises a light chain sequence as set forth in SEQ ID NO:9 and a heavy chain sequence as set forth in SEQ ID NO: 10.

30. The method of any preceding claim, wherein the anti-IL-33 antibody variant has the same pharmacokinetic (pK) characteristics as tozorakimab in humans.

31. The method of any preceding claim, wherein the anti-IL-33 antibody is tozorakimab.

32. The method according to any preceding claim, wherein the administration is subcutaneous.

33. The method of any preceding claim, wherein the anti-IL-33 antibody or antibody variant thereof is administered for a period of at least 12 weeks.

34. The method of any preceding claim, wherein the anti-IL-33 antibody or antibody variant thereof is administered for a period of at least 24 weeks.

35. The method of any preceding claim, wherein the anti-IL-33 antibody or antibody variant thereof is administered for a period of at least 52 weeks.

36. The anti-IL-33 antibody or antibody variant thereof characterised in any preceding claim for use in a method of treating asthma, wherein the method is that characterised in any preceding claim.

37. Use of the anti-IL-33 antibody or antibody variant thereof characterised in any of claims 1 to 36, in the manufacture of a medicament for use in a method of treating asthma characterised in any of claims 1 to 36.


SEQUENCE LISTING (11 sequences)

SEQ ID NO: 1 - protein, 5 aa, synthetic construct
  source 1..5: note = Tozorakimab VHCDR1
SYAMS

SEQ ID NO: 2 - protein, 17 aa, synthetic construct
  source 1..17: note = Tozorakimab VHCDR2
GISAIDQSTYYADSVKG

SEQ ID NO: 3 - protein, 17 aa, synthetic construct
  source 1..17: note = Tozorakimab VHCDR3
QKFMQLWGGGLRYPFGY

SEQ ID NO: 4 - protein, 126 aa, synthetic construct
  source 1..126: note = Tozorakimab VH Domain
EVQLLESGGGLVQPGGSLRLSCAASGFTFSSYAMSWVRQAPGKGLEWVSGISAIDQSTYYADSVKGRFTISRDNSKNTLYLQMNSLRAEDTAVYYCARQKFMQLWGGGLRYPFGYWGQGTMVTVSS

SEQ ID NO: 5 - protein, 11 aa, synthetic construct
  source 1..11: note = Tozorakimab VLCDR1
SGEGMGDKYAA

SEQ ID NO: 6 - protein, 7 aa, synthetic construct
  source 1..7: note = Tozorakimab VLCDR2
RDTKRPS

SEQ ID NO: 7 - protein, 9 aa, synthetic construct
  source 1..9: note = Tozorakimab VLCDR3
GVIQDNTGV

SEQ ID NO: 8 - protein, 106 aa, synthetic construct
  source 1..106: note = Tozorakimab VL Domain
SYVLTQPPSVSVSPGQTASITCSGEGMGDKYAAWYQQKPGQSPVLVIYRDTKRPSGIPERFSGSNSGNTATLTISGTQAMDEADYYCGVIQDNTGVFGGGTKLTVL

SEQ ID NO: 9 - protein, 212 aa, synthetic construct
  source 1..212: note = Tozorakimab light chain
SYVLTQPPSVSVSPGQTASITCSGEGMGDKYAAWYQQKPGQSPVLVIYRDTKRPSGIPERFSGSNSGNTATLTISGTQAMDEADYYCGVIQDNTGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - protein, 456 aa, synthetic construct
  source 1..456: note = Tozorakimab heavy chain
EVQLLESGGGLVQPGGSLRLSCAASGFTFSSYAMSWVRQAPGKGLEWVSGISAIDQSTYYADSVKGRFTISRDNSKNTLYLQMNSLRAEDTAVYYCARQKFMQLWGGGLRYPFGYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 270 aa, Homo sapiens
  source 1..270: note = IL-33 full length protein
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET